SEQUENCE LISTING (43 sequences)

SEQ ID NO: 1 - DNA, 46 bp, synthetic construct
  misc_feature 1..46: note = Synthetic construct
  misc_feature 43: note = n = U
  source 1..46: mol_type = other DNA
ccacgacgacgacgacgagcgttaggctcgtcgtcgtcgtcgnggt

SEQ ID NO: 2 - DNA, 11 bp, synthetic construct
  misc_feature 1..11: note = Synthetic construct
  source 1..11: mol_type = other DNA
ttagcgttagt

SEQ ID NO: 3 - DNA, 11 bp, synthetic construct
  misc_feature 1..11: note = Synthetic construct
  source 1..11: mol_type = other DNA
ctagcgtcagt

SEQ ID NO: 4 - DNA, 17 bp, synthetic construct
  misc_feature 1..17: note = Synthetic construct
  source 1..17: mol_type = other DNA
actgacgctagtgctag

SEQ ID NO: 5 - DNA, 17 bp, synthetic construct
  misc_feature 1..17: note = Synthetic construct
  source 1..17: mol_type = other DNA
tgactgcgatcacgatc

SEQ ID NO: 6 - DNA, 18 bp, synthetic construct
  misc_feature 1..18: note = Synthetic constrct
  source 1..18: mol_type = other DNA
actgacgctagtgctaga

SEQ ID NO: 7 - DNA, 18 bp, synthetic construct
  misc_feature 1..18: note = Synthetic constrct
  source 1..18: mol_type = other DNA
atgactgcgatcacgatc

SEQ ID NO: 8 - DNA, 28 bp, synthetic construct
  misc_feature 1..28: note = Synthetic constrct
  source 1..28: mol_type = other DNA
  modified_base 2: mod_base = OTHER; note = uracil
gtggtactgacgctagtgctagaaccac

SEQ ID NO: 9 - DNA, 27 bp, synthetic construct
  misc_feature 1..27: note = Synthetic constrct
  source 1..27: mol_type = other DNA
  modified_base 26: mod_base = OTHER; note = uracil
caccatgactgcgatcacgatctggtg

SEQ ID NO: 10 - DNA, 26 bp, synthetic construct
  misc_feature 1..26: note = Synthetic constrct
  source 1..26: mol_type = other DNA
gggtactgacgctagtgctagaccac

SEQ ID NO: 11 - DNA, 26 bp, synthetic construct
  misc_feature 1..26: note = Synthetic constrct
  source 1..26: mol_type = other DNA
caccatgactgcgatcacgatctggg

SEQ ID NO: 12 - DNA, 17 bp, synthetic construct
  misc_feature 1..17: note = Synthetic constrct
  source 1..17: mol_type = other DNA
gtggtactgacgctaga

SEQ ID NO: 13 - DNA, 11 bp, synthetic construct
  misc_feature 1..11: note = Synthetic constrct
  source 1..11: mol_type = other DNA
tgctagaccac

SEQ ID NO: 14 - DNA, 16 bp, synthetic construct
  misc_feature 1..16: note = Synthetic constrct
  source 1..16: mol_type = other DNA
caccatgactgcgatc

SEQ ID NO: 15 - DNA, 12 bp, synthetic construct
  misc_feature 1..12: note = Synthetic constrct
  source 1..12: mol_type = other DNA
aacgatctggtg

SEQ ID NO: 16 - DNA, 17 bp, synthetic construct
  misc_feature 1..17: note = Synthetic constrct
  source 1..17: mol_type = other DNA
gtggtactgacgctaga

SEQ ID NO: 17 - DNA, 12 bp, synthetic construct
  misc_feature 1..12: note = Synthetic constrct
  source 1..12: mol_type = other DNA
ttgctagaccac

SEQ ID NO: 18 - DNA, 17 bp, synthetic construct
  misc_feature 1..17: note = Synthetic constrct
  source 1..17: mol_type = other DNA
caccatgactgcgatct

SEQ ID NO: 19 - DNA, 12 bp, synthetic construct
  misc_feature 1..12: note = Synthetic constrct
  source 1..12: mol_type = other DNA
aacgatctggtg

SEQ ID NO: 20 - DNA, 34 bp, synthetic construct
  misc_feature 1..34: note = Synthetic constrct
  source 1..34: mol_type = other DNA
  modified_base 2: mod_base = OTHER; note = uracil
  modified_base 9: mod_base = OTHER; note = uracil
  modified_base 14..15: mod_base = OTHER; note = uracil
  modified_base 17: mod_base = OTHER; note = uracil
tttagcgttagtattatgtggtactgacgctaga

SEQ ID NO: 21 - DNA, 24 bp, synthetic construct
  misc_feature 1..24: note = Synthetic constrct
  modified_base 4..5: mod_base = OTHER; note = uracil
  modified_base 7: mod_base = OTHER; note = uracil
  modified_base 23: mod_base = OTHER; note = uracil
  source 1..24: mol_type = other DNA
agattatgtggtctagcaattgtt

SEQ ID NO: 22 - DNA, 12 bp, synthetic construct
  source 1..12: mol_type = other DNA
ccgtcggaccgc

SEQ ID NO: 23 - DNA, 12 bp, synthetic construct
  source 1..12: mol_type = other DNA
ggcagcctggcg

SEQ ID NO: 24 - DNA, 12 bp, synthetic construct
  source 1..12: mol_type = other DNA
ccgtcggaccgc

SEQ ID NO: 25 - DNA, 12 bp, synthetic construct
  source 1..12: mol_type = other DNA
ggcagcctggcg

SEQ ID NO: 26 - DNA, 12 bp, synthetic construct
  source 1..12: mol_type = other DNA
ccgtcggaccgc

SEQ ID NO: 27 - DNA, 12 bp, synthetic construct
  source 1..12: mol_type = other DNA
ggcagcctggcg

SEQ ID NO: 28 - DNA, 12 bp, synthetic construct
  misc_feature 1..2: note = RNA
  misc_feature 3..8: note = DNA
  misc_feature 9: note = RNA
  misc_feature 10..11: note = DNA
  misc_feature 12: note = RNA
  source 1..12: mol_type = other DNA
ttgtcggatcgt

SEQ ID NO: 29 - DNA, 12 bp, synthetic construct
  misc_feature 1..2: note = DNA
  misc_feature 3: note = RNA
  misc_feature 4..5: note = DNA
  misc_feature 6..7: note = RNA
  misc_feature 8..12: note = DNA
  source 1..12: mol_type = other DNA
ggtagtttggcg

SEQ ID NO: 30 - DNA, 12 bp, synthetic construct
  source 1..12: mol_type = other DNA
ccgtcggaccgc

SEQ ID NO: 31 - DNA, 12 bp, synthetic construct
  source 1..12: mol_type = other DNA
ttgtcggatcgt

SEQ ID NO: 32 - DNA, 12 bp, synthetic construct
  source 1..12: mol_type = other DNA
gcggtttgatgg

SEQ ID NO: 33 - DNA, 29 bp, synthetic construct
  source 1..29: mol_type = other DNA
ccgtcggaccgcnnnnnnnnnnnnnnnnn

SEQ ID NO: 34 - skipped

SEQ ID NO: 35 - DNA, 37 bp, synthetic construct
  source 1..37: mol_type = other DNA
ccgtcggaccgcnnnnnnnnnnnnnnnnnnnnnnnnn

SEQ ID NO: 36 - skipped

SEQ ID NO: 37 - DNA, 45 bp, synthetic construct
  source 1..45: mol_type = other DNA
ccgtcggaccgcnnnnnnnnnnnnnnnnnnnnnnnnnnnnnnnnn

SEQ ID NO: 38 - skipped

SEQ ID NO: 39 - DNA, 29 bp, synthetic construct
  source 1..29: mol_type = other DNA
ggcagcctggcgnnnnnnnnnnnnnnnnn

SEQ ID NO: 40 - DNA, 12 bp, synthetic construct
  source 1..12: mol_type = other DNA
gcggtccgacgg

SEQ ID NO: 41 - DNA, 12 bp, synthetic construct
  source 1..12: mol_type = other DNA
ttgtcggatcgt

SEQ ID NO: 42 - DNA, 12 bp, synthetic construct
  source 1..12: mol_type = other DNA
ccgtcggaccgc

SEQ ID NO: 43 - DNA, 12 bp, synthetic construct
  source 1..12: mol_type = other DNA
gcggtttgatgg

(19) United States
(12) Patent Application Publication
(10) Pub. No.: US 2025/0361553 A1
(43) Pub. Date: Nov. 27, 2025

(54) Methods and Compositions for the Simultaneous Identification and Mapping of DNA Methylation

(71) Applicant: New England Biolabs, Inc., Ipswich, MA (US)

(72) Inventors: Bo Yan, Ipswich, MA (US); Zhiyi Sun, Winchester, MA (US); Romualdas Vaisvila, Ipswich, MA (US); Laurence Ettwiller, Beverly, MA (US); Louise J. Williams, Reading, MA (US); Chaithanya Ponnaluri, Ipswich, MA (US); Daniel J. Evanich, Gloucester, MA (US); Vaishnavi Panchapakesa, Newton, MA (US)

(73) Assignee: New England Biolabs, Inc., Ipswich, MA (US)

(21) Appl. No.: 18/874,715

(22) PCT Filed: Jun. 14, 2023
(86) PCT No.: PCT/US2023/068429

§ 371 Date: Dec. 13, 2024

Related U.S. Application Data
(60) Provisional application No. 63/366,343, filed on Jun. 14, 2022.
(60) Provisional application No. 63/366,340, filed on Jun. 14, 2022.
(60) Provisional application No. 63/399,970, filed on Aug. 22, 2022.

(51) Int. Cl. C12Q 1/6869; C12Q 1/34; C12Q 1/48; C12Q 1/6855; C12Q 1/6876

(52) U.S. Cl. CPC C12Q 1/6869; C12Q 1/34; C12Q 1/48; C12Q 1/6855; C12Q 1/6876; C12Y 207/07007; C12Y 301/00; C12Y 305/04001; C12Q 2600/154; C12Q 2600/156

(57) ABSTRACT

Provided herein is a method for generating a strand of DNA. In some embodiments, this method may comprise: (a) ligating a hairpin adaptor to a double-stranded fragment of DNA to produce a ligation product; (b) enzymatically generating a free 3′ end in a double-stranded region of the hairpin adaptor in the ligation product; and (c) extending the free 3′ end in a dCTP-free reaction mix that comprises a strand-displacing or nick-translating polymerase, dGTP, dATP, dTTP and modified dCTP to generate a hairpin product that has an original strand and a neosynthesized strand that contains modified Cs.

### CROSS-REFERENCING

This application claims the benefit of U.S. Provisional Application Ser. Nos. 63/366,343, filed on Jun. 14, 2022; 63/366,340, filed on Jun. 14, 2022; and 63/399,970, filed on Aug. 22, 2022, which applications are incorporated by reference herein.

### SEQUENCE LISTING

A Sequence Listing is provided herewith as a Sequence Listing XML, “NEB-461-PCT.xml” created on Jun. 14, 2023, and having a size of 50.5 KB. The contents of the Sequence Listing XML are incorporated by reference herein in their entirety.

### BACKGROUND

The covalent modification of cytosine by a methyl group leads to the formation of 5-methylcytosine (5mC), a key epigenetic modification of genomic DNA that occurs in a large number of organisms and represents so far the best characterized form of DNA modification. In mammals, patterns of methylation are established early during embryogenesis and include X-chromosome inactivation, imprinting, and the repression of repeats and transposable elements (Greenberg and Bourc'his 2019). Not surprisingly, global or regional changes of DNA methylation are among the earliest events known to occur in cancer (Baylin and Jones 2016). The identification of methylation profiles in humans is a key step in studying disease processes and is increasingly used for diagnostic purposes.

In prokaryotes, the vast majority of genomes contain 5mC (Blow et al. 2016). Contrary to eukaryotes where the methylation sites are variable and subject to epigenetic states, bacterial methylations tend to be constitutively present at specific sites across the genome. These sites are defined by the methylase specificity and, in the case of RM systems, tend to be fully methylated to avoid cuts by the cognate restriction enzyme. Current high throughput techniques for the identification of 5mC using Illumina sequencing is performed by converting cytosine to uracil, leaving 5′methylcytosine (5mC) intact. This conversion is done using chemical treatment (bisulfite) or enzymatic treatment (EM-seq). In any case, this conversion must be complete, leading most of the time to the separation of the two DNA strands and a sharp reduction of genome sequence complexity from 4 to essentially 3 nucleotides with thymine (T) being either the product of amplification after deamination of C or of a genuine T.

Consequently, identification of methylation requires specialized technologies, specialized analysis pipelines and a reference genome. Any additional information such as sequence or variation is essentially lost and would require additional experiments to obtain them. Recently, a new technique has been developed that locks Watson and Crick strand together by hairpin adaptor followed by bisulfite treatment (Liang et al. 2021). However, because both strands are subjected to conversion in the Liang method, none of the strands retains the 4-letter code and, as such, potential information is lost in the process. This disclosure solves this problem and others.

### SUMMARY

Provided herein is a method for generating a deamination-resistant strand of DNA. In some embodiments, the method may comprise: (a) ligating a hairpin adaptor to a double-stranded fragment of DNA to produce a ligation product; (b) enzymatically generating a free 3′ end in a double-stranded region of the hairpin adaptor in the ligation product; and (c) extending the free 3′ end in a dCTP-free reaction mix that comprises a strand-displacing or nick-translating polymerase, dGTP, dATP, dTTP and modified dCTP to generate a hairpin product that has an original strand and a neosynthesized strand that contains modified Cs. In some embodiments, the method may comprise (d) deaminating the hairpin product or an adaptor-ligated product thereof, wherein the modified Cs protect the neosynthesized strand from deamination. The method may further include (d) deaminating the hairpin product or an adaptor-ligated product thereof, wherein the modified Cs protect the neosynthesized strand from deamination.

In an embodiment, the deaminating is done using bisulfite. In an embodiment, the deaminating is done using a cytosine deaminase, optionally after enzymatically protecting any modified Cs in the original strand from deamination. The cytosine deaminase may modify a double-stranded or single-stranded substrate. In an embodiment, the method may further comprise amplifying the deaminated product of step (d) thereby converting any deaminated Cs to Ts in the amplification product.

In an embodiment, the methods are used for enriching target molecules using a probe that is complementary to a sequence in the double-stranded fragment of (a).

The methods may further include sequencing the deaminated product, or an amplification product thereof, to produce sequence. In an embodiment, the methods involve identifying a C in the sequence corresponding to the original strand, wherein the C corresponds to a modified cytosine. The methods may further involve mapping the modified cytosine to a site in a reference genome and annotating the site as being modified.

In embodiments of the disclosed methods, the modified dCTP may be dmCTP, pyrrolo-dCTP or N4-dmCTP.

In an embodiment, the double-stranded fragment of DNA may be a fragment of mammalian DNA; in an embodiment, the double-stranded fragment of DNA is a molecule of cfDNA.

In embodiments of the disclosed methods, methods may include enzymatically modifying the double-stranded fragment of DNA, the ligation product or hairpin product to protect any modified cytosines or hydroxymethylcytosines from deamination.

In embodiments, in step (a) both ends of the double-stranded fragment of DNA are ligated to the hairpin adaptor and in step (b) the top and bottom strands of the double-stranded fragment of DNA become separated.

In an embodiment, step (b) is done using USER, an endonuclease, a nicking endonuclease or an RNase.

In various embodiments, the hairpin adaptor has at least one modified C and no Cs. In an embodiment, the modified C of the adaptor is mCTP, pyrrolo-CTP or N4-mCTP.

Provided herein are reaction mixes. In an embodiment, a reaction mix includes: (a) a hairpin DNA that has a free 3′ end in a double-stranded region; (b) a strand-displacing or nick-translating polymerase, and (c) dGTP, dATP, dTTP, modified dCTP and no dCTP. In an embodiment, the hairpin DNA comprises a fragment of mammalian DNA ligated to a hairpin adaptor. In an embodiment, the hairpin DNA comprises a molecule of cfDNA ligated to a hairpin adaptor. In an embodiment, the modified dCTP may be dmCTP, pyrrolo-dCTP or N4-dmCTP.

Provided herein are nucleic acid molecules. In an embodiment, a nucleic acid molecule contains, in order from 5′ to 3′: a first sequence, a linker, and a second sequence, wherein: the first sequence is composed of Gs, As, Ts, Cs and modified Cs; the second sequence is composed of Gs, As, Ts, modified Cs and no Cs; and the first and second sequences are complementary. In another embodiment, a nucleic acid molecule contains, in order from 5′ to 3′: a first sequence, a linker, and a second sequence, wherein: the first sequence is composed of Gs, As, Ts, Us and modified Cs and the second sequence is composed of Gs, As, Ts, modified Cs and no Cs; and the first and second sequences are complementary except for the Us in the first sequence.

Provided herein are kits for generating a deamination-resistant strand of DNA. In an embodiment, a kit includes: (a) a hairpin adaptor containing a U in a double-stranded region of the adaptor; (b) one or more enzymes that create a nick at the site of the U; (c) a modified dCTP; and (d) a nick-translating or strand-displacing polymerase. In an embodiment, the modified dCTP may be dmCTP, pyrrolo-dCTP or N4-dmCTP. In an embodiment, the adaptor contains modified Cs and no Cs. In an embodiment, the modified Cs of the adaptor may be mCTP, pyrrolo-CTP or N4-mCTP. A kit may further include a deaminase, wherein the modified Cs are deamination resistant.

Also provided are methods for generating a deamination-resistant strand of DNA using one hairpin. The method involves (a) separating the strands of a double-stranded fragment of DNA to produce a single-stranded fragment; (b) attaching a double-stranded adaptor to the 3′ end of the single-stranded fragment; (c) extending the free 3′ end of an attached double-stranded adaptor in a dCTP-free reaction mix that comprises a strand-displacing or nick-translating polymerase; and dGTP, dATP, dTTP, and modified dCTP, to generate a double-stranded product; (d) attaching a hairpin adaptor to the 5′ end of the double-stranded product to generate a hairpin product that has an original strand and a neosynthesized strand that contains modified Cs.

### DESCRIPTION OF FIGURES

The skilled artisan will understand that the drawings, described below, are for illustration purposes only. The drawings are not intended to limit the scope of the present teachings in any way.

FIGS. 1A and 1B: Overview of Methyl-SNP-seq: FIG. 1A: Experimental workflow of Methyl-SNP-seq: 1—the genomic DNA is fragmented to ˜400bp fragments. 2—Hairpin adaptors are ligated at both ends of the fragmented DNA, forming a dumbbell shaped DNA. Next, nicks at both opposite ends of the adaptors are introduced and using nick translation, a copy of the original strand is synthesized replacing CTP as a source of nucleotide with m5CTP instead. This nick translation step broke the dumbbell shaped DNA somewhere in the middle of the fragment. Fragments are now on average ˜200bp long. 3-Methylated Illumina Y-shaped adaptors are ligated to the blunt-ends. 4-bisulfite conversion opens the DNA structure revealing a single strand DNA molecule that can be amplified using the Illumina adaptors. Sequencing requires paired-end reads to obtain both the methylation and the genomic sequence information (Materials and Methods). For more details on the experimental procedure, see FIG. 2A. FIG. 1B: Deconvolution procedure. For more details on the bioinformatics analysis, see FIG. 2B.

FIGS. 2A and 2B: Detailed description of the Methyl-SNP-seq experimental workflow (FIG. 2A) and flowchart illustration of the analysis of Human Methyl-SNP-seq data (FIG. 2B). R1 and R2 stand for Read1 and Read2.

FIGS. 3A-3C show a comparison of SNP calling with different strategies. The defined SNPs were benchmarked against JIMB WGS data. FIG. 3A: Methyl-SNP-seq replicate 1 data was used for SNP calling using: Reference-free Deconvoluted Read and Reference-dependent Deconvoluted Read. FIG. 3B: Characterization of the False positive heterozygous SNPs defined by Deconvolued Read or Read2. (i.e. T-C means in the vcf file REF=T while ALT=C). FIG. 3C: Precision of SNPs defined using Deconvoluted Read and Read2. The common SNPs are those detected by both Deconvoluted Read and Read2.

FIGS. 4A-4E show SNP identification by Methyl-SNP-seq. The JIMB whole genome sequencing of NA12878 was used as a benchmark for comparison. FIG. 4A: Comparison of SNPs identified using Methyl-SNP-seq Deconvoluted Read and Read2 with those using JIMB whole genome sequencing data. Common SNPs, which were identified by both Deconvoluted Read and Read2 and marked by red dashed lines, are referred to as Methyl-SNP-seq defined SNPs. FIG. 4B: Precision and Sensitivity of SNP identification using different numbers of Methyl-SNP-Seq reads. Precision=TP/(TP+FP). Sensitivity=TP/(TP+FN) with TP: True positive. FP: False positive. FN: False negative. FIG. 4C: Fraction of heterozygous and homozygous Methyl-SNP-seq defined SNPs. FIG. 4D: Distribution of the genome coverage of the False Negative SNP sites. FIG. 4E: Characterization of the JIMB and True Positive Methyl-SNP-seq defined SNPs.

FIGS. 5A-5D show methylome data. FIG. 5A: Pairwise comparison of methylation level of CpG islands measured by Methyl-SNP-seq, whole genome bisulfite sequencing by ENCODE (WGBS) and Nanopore sequencing. Each dot represents a CpG island. Only CpG islands having coverage>=50 were used for correlation calculation. There are 27050, 27313 and 16071 CpG islands detected by Methyl-SNP-seq, WGBS and Nanopore sequencing, respectively. FIG. 5B: The genome coverage of Methyl-SNP-seq and WGBS on chr2. FIG. 5C Distribution (kde plot) of % methylation on CpG sites having coverage>=5. FIG. 5D: Fraction of coverage on CpG sites.

FIG. 6 shows a schematic of a process for generating a sequence library for obtaining both the methylation and the genomic sequence information in which a hairpin adaptor is added on one end of the DNA to be sequenced.

FIGS. 7A-7C shows schematics of configurations of a single stranded DNA fragment annealed to an adaptor (FIG. 7A); an adaptor including a known UMI and a random sequence (FIG. 7B); and an adaptor including a random UMI, known index sequence, and random sequence (FIG. 7C).

FIG. 8A shows a schematic of a double stranded DNA containing an original strand and a neosynthesized strand, which is attached to an adaptor. FIG. 8B shows a schematic of a double stranded DNA containing an original strand and a neosynthesized strand, which is attached to a 3′ adaptor and a 5′ hairpin adaptor.

FIG. 9 shows a schematic of linear DNAs containing the original (epigenetic) sequence information (OT=top strand; OB=bottom strand) and neosynthesized (genetic) sequence information (CTOT=top strand; CTOB=bottom strand).

### DETAILED DESCRIPTION

Provided herein is a method for generating a deamination-resistant strand of DNA. In some embodiments, the method may comprise: (a) ligating a hairpin adaptor to a double-stranded fragment of DNA to produce a ligation product; (b) enzymatically generating a free 3′ end in a double-stranded region of the hairpin adaptor in the ligation product; and (c) extending the free 3′ end in a dCTP-free reaction mix that comprises a strand-displacing or nick-translating polymerase, dGTP, dATP, dTTP and modified dCTP to generate a hairpin product that has an original strand and a neosynthesized strand that contains modified Cs. In some embodiments, the method may comprise: (d) deaminating the hairpin product or an adaptor-ligated product thereof, wherein the modified Cs protect the neosynthesized strand from deamination.

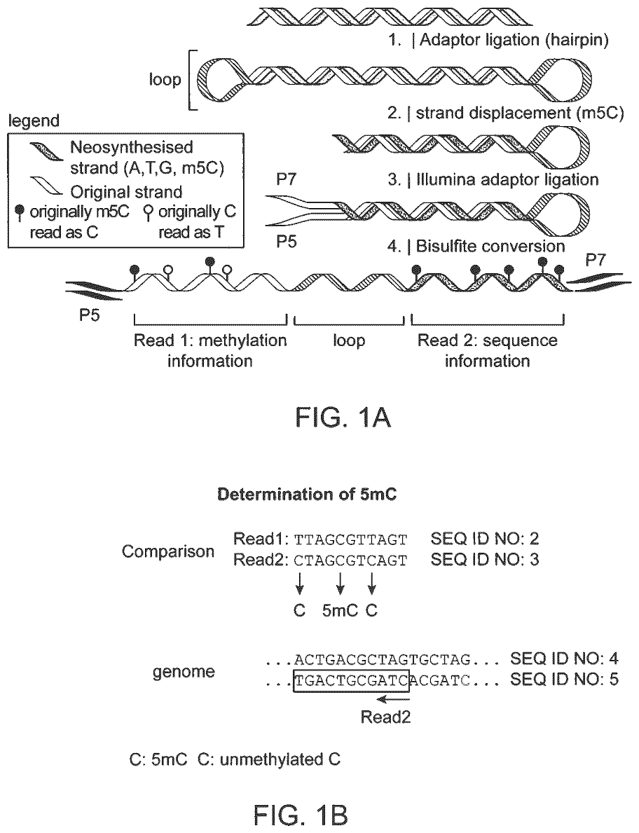

Because the top and bottom strands of the double stranded molecule are locked together by a hairpin, the neosynthesized strand (which provides the “sequence information”) and the deaminated strand (which provides the “methylation information”) can be read on the same paired-end read. The sequence of the neosynthesized strand provides an internal reference for the deaminated strand, thereby allowing methylated cytosines to be identified by comparing the sequence of the neosynthesized strand to the sequence of the deaminated strand in a pair of paired- end reads (see FIGS. 1A and B), without a reference sequence (e.g., a reference genome). In addition, the neosynthesized strand retains the four letter (G, A, T, C) code, thereby allowing sequence variations (e.g., SNPs) and methylated cytosines to be readily identified in the same molecule. Thus, in using the present method, the interplay between sequence variations and methylation can be analyzed at a single molecule resolution. Finally, because the neosynthesized strand contains the original four letter (G, A, T, C) code, fragments from a library produced by the present method can be enriched using conventional probes that are designed using genomic sequence as a template.

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and ease of reference.

Sources of commonly understood terms and symbols may include: standard treatises and texts such as Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

As used herein and in the appended claims, the singular forms “a”, “an”, and “the” include plural referents unless the context clearly dictates otherwise. For example, the term “a protein” refers to one or more proteins, i.e., a single protein and multiple proteins. The claims can be drafted to exclude any optional element when exclusive terminology is used such as “solely,” “only” are used in connection with the recitation of claim elements or when a negative limitation is specified.

Aspects of the present disclosure can be further understood in light of the embodiments, section headings, figures, descriptions and examples, none of which should be construed as limiting the entire scope of the present disclosure in any way. Accordingly, the claims set forth below should be construed in view of the full breadth and spirit of the disclosure.

Each of the individual embodiments described and illustrated herein has discrete components and features which may be readily separated from or combined with the features of any of the other several embodiments without departing from the scope or spirit of the present teachings. Any recited method can be carried out in the order of events recited or in any other order which is logically possible.

Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample numerical values are provided, each alone may represent an intermediate value in a range of values and together may represent the extremes of a range unless specified.

In the context of the present disclosure, “non-naturally occurring” refers to a polynucleotide, polypeptide, carbohydrate, lipid, or composition that does not exist in nature. Such a polynucleotide, polypeptide, carbohydrate, lipid, or composition may differ from naturally occurring polynucleotides polypeptides, carbohydrates, lipids, or compositions in one or more respects. For example, a polymer (e.g., a polynucleotide, polypeptide, or carbohydrate) may differ in the kind and arrangement of the component building blocks (e.g., nucleotide sequence, amino acid sequence, or sugar molecules). A polymer may differ from a naturally occurring polymer with respect to the molecule(s) to which it is linked. For example, a “non-naturally occurring” protein may differ from naturally occurring proteins in its secondary, tertiary, or quaternary structure, by having a chemical bond (e.g., a covalent bond including a peptide bond, a phosphate bond, a disulfide bond, an ester bond, and ether bond, and others) to a polypeptide (e.g., a fusion protein), a lipid, a carbohydrate, or any other molecule. Similarly, a “non-naturally occurring” polynucleotide or nucleic acid may contain one or more other modifications (e.g., an added label or other moiety) to the 5′-end, the 3′ end, and/or between the 5′- and 3′-ends (e.g., methylation) of the nucleic acid. A “non-naturally occurring” composition may differ from naturally occurring compositions in one or more of the following respects: (a) having components that are not combined in nature; (b) having components in concentrations not found in nature; (c) omitting one or components otherwise found in naturally occurring compositions; (d) having a form not found in nature, e.g., dried, freeze dried, crystalline, aqueous; and (e) having one or more additional components beyond those found in nature (e.g., buffering agents, a detergent, a dye, a solvent or a preservative).

In the context of the present disclosure, “modified cytosine” refers to any covalent modification of cytosine including naturally occurring and non-naturally occurring modifications. Modified cytosines include, for example, 1-methylcytosine (1mC), 2-O-methylcytosine (m2C), 3-ethylcytosine (e3C), 3,N⁴-ethylenocytosine (εC), 3-methylcytosine (3mC), 4-methylcytosine (4mC), 5-carboxylcytosine (5CaC), 5-formylcytosine (5fC), 5-hydroxymethylcytosine (5hmC), 5-methylcytosine (5mC), N⁴-methylcytosine (N4mC), 5-carbamoyloxymethylcytosine, 5-(beta-D-glucosylmethyl)cytosine, pyrrolo-cytosine (pyrrolo-C). 5-carboxylcytosine (5caC) is the final oxidized derivative of 5-methylcytosine (5mC). 5mC is oxidized to 5-hydroxymethylcytosine (5hmC) which is then oxidized to 5-formylcytosine (5fC) then 5caC. Additional examples of modified nucleotides may be found at https://dnamod.hoffmanlab.org and Parker, M. J., Lee, Y.-J., Weigele, P. R. & Saleh, L. (2020). 5-Methylpyrimidines and their modifications in DNA. In Comprehensive Natural Products ///(pp. 465-488). Elsevier.

In some embodiments, a method may involve use of a double-stranded DNA substrate referenced as a double-stranded fragment of DNA. Such DNA substrates may have a length of ≤50nucleotides, 10-200 nucleotides, 80-400 nucleotides, 50-500 nucleotides, ≤500nucleotides, or larger depending on the sequencing technology selected. In some embodiments, the DNA substrate may be a fragment of genomic DNA, organelle DNA, cDNA, cell free DNA (cfDNA), or other DNAs of interest and can be or arise from any desired source (e.g., human, non-human mammal, plants, insects, microbial, viral, or synthetic DNA). A DNA substrate may be prepared, in some embodiments by extracting (e.g., genomic DNA) from a biological sample and, optionally, fragmenting it. In some embodiments, fragmenting DNA may comprise mechanically fragmenting the DNA (e.g., by sonication, nebulization, or shearing) or enzymatically fragmenting the DNA (e.g., using a double stranded DNA “dsDNA” fragmentation mix). Examples of enzymes for fragmentation include NEBNext® Fragmentase®, UltraShear™, and FS systems (New England Biolabs, Ipswich MA), among others. A DNA substrate may be already fragmented (e.g., as is the case for FFPE samples and circulating cell-free DNA (cfDNA)).

A method may include polishing DNA ends (e.g., the ends of fragmented DNA). For example, DNA ends may be contacted with (a) a proofreading polymerase to excise 3′ overhanging nucleotides, if any, (b) a proofreading and/or non-proofreading polymerase to fill in 5′ overhangs, if any, and/or (c) a polynucleotide kinase (PNK) to phosphorylate unphosphorylated 5′ ends, if any. A method may comprise contacting DNA ends (e.g., blunt ends) with a non-proofreading polymerase to add an untemplated A-tail (e.g., a single base overhang comprising adenine) to the 3′ end. Methods may include ligating one or more adaptors to DNA ends. Adaptors may comprise one or more sample tags, unique molecular identifiers (UMIs), modified nucleotides, primer sequences (e.g., for sequencing). In some embodiments, adaptors may comprise cytosines that are not substrates for the deaminase to be used. If desired, polishing products and/or ligation products may be cleaned up, for example, to separate polishing products or ligation products, as applicable, from enzymes, unreacted nucleotides and/or adaptors.

All publications, patents, and patent applications mentioned in this specification are herein incorporated by reference to the same extent as if each individual publication, patent, or patent application was specifically and individually indicated to be incorporated by reference including U.S. Provisional Application Ser. Nos. 63/366,340 filed Jun. 14, 2022; 63/366,340, filed on Jun. 14, 2022, and 63/399,970, filed on Aug. 22, 2022, which applications are incorporated by reference herein.

This disclosure encompasses methods, compositions and kits that are here referred to as “Methyl-SNP-Seq” as well as related methods. Some of the principles of the method are illustrated in FIGS. 1A and 1B. As illustrated, the method may be used to generate a deamination-resistant strand of DNA. In these embodiments, the method may comprise: ligating a hairpin adaptor to a double-stranded fragment of DNA to produce a ligation product, enzymatically generating a free 3′ end in a double-stranded region of the hairpin adaptor in the ligation products, and extending the free 3′ end in a dCTP-free reaction mix that comprises a strand-displacing or nick-translating polymerase, dGTP, dATP, dTTP and modified dCTP to generate a hairpin product that has an original strand and a neosynthesized strand that contains modified Cs. In these embodiments, the modified Cs that are incorporated into the neosynthesized strand make the neosynthesized strand deamination resistant.

Because this reaction is initiated at a gap by a strand-displacing or nick-translating polymerase, it is not a gap-fill reaction and there is no ligation that seals the ends of a newly synthesized strand and another strand. As such, the extension step is performed in the absence of a ligase.

As reflected by the description herein, a “modified dCTP” can be incorporated by a polymerase into a neosynthesized strand and is distinct from dCTP in that it has a chemical structure that is not converted to uracil or another moiety under deaminating conditions. As a result, the sequence of the neosynthesized strand reflects the genetic sequence of the DNA substrate rather than the epigenetic sequence.

As illustrated in FIG. 1A, in some embodiments, the method may comprise deaminating the hairpin product before or after it is ligated to an adaptor. The modified Cs protect the neosynthesized strand from deamination. The deamination step (step 3 in FIG. 1A) can be done chemically or enzymatically. For example, the deaminating may be done using bisulfite (as illustrated) or using a cytosine deaminase (see, generally, Sun et al, Genome Res. 2021 31: 291-300 and Vaisvila et al Genome Res. 2021 31: 1280-1289), where the cytosine deaminase could recognize single-stranded or double-stranded DNA molecules. In some embodiments, induced cytidine deaminase (AID) or an APOBEC enzyme APOBEC-1 (Apo1), APOBEC-2 (Apo2), AID, APOBEC-3A, -3B, -3C, -3DE, -3F, -3G, -3H or APOBEC-4 (Apo4) could be used. Any of these enzymes could be used in conjunction with a gyrase, for example.

If a double-stranded deaminase is used, the deaminase may be any of the deaminases described in WO 2023/097226, published Jun. 1, 2023, which claims priority to 63/264,513, filed on Nov. 24, 2021 (e.g., the deaminases referred to MGYP001104162829, RaDa01, LbsDa01, CseDa01, CrDa01, d38_MGY29, among many others), which application is incorporated by reference herein.

In some of these embodiments (and depending on which deaminase is used) the modified Cs in the original strand may themselves be enzymatically modified to make them deaminase resistant, thereby allowing the modified Cs in the original strand to stay as Cs in the sequence reads. This protection step may be done by treating the ligation product with TET (e.g., TET2) and/or BGT (DNA beta-glucosyltransferase) before deamination (see, e.g., Sun et al, supra, Vaisvila et al supra and Schutsky et al Nucleic Acids Research 2017 45, among others). Depending on how the deamination is going to be done, the modified dCTP could be dmCTP (which is bisulfite resistant), pyrrolo-dCTP, or N⁴-dmCTP (which are deaminase-resistant), although other modified dCTPs could be used. Any Cs in the adaptor sequence may be deamination resistant too and, in some embodiments, may be mCTP, pyrrolo-CTP or N⁴-mCTP, for example. When using a deamination reaction that converts modified cytosine to T (e.g., a deaminase having specificity for modified cytosines, such as 5mC and/or 5hmC), the method may employ dCTP rather than modified dCTP when extending the free 3′ end in a reaction mix that comprises a strand-displacing or nick-translating translating polymerase to generate a hairpin product that has an original strand and a neosynthesized strand that contains modified Cs.

As illustrated in FIG. 1A, after the sample has been deaminated, the method may further comprise amplifying the deaminated product of step (d) thereby converting any deaminated Cs in the original strand to Ts in the amplification product. As illustrated, this may be done by ligating an asymmetric (or “Y”) adaptor, e.g., an Illumina P5/P7 adaptor, onto the deaminated product and then amplifying the deaminated product using primers that correspond to the sequences in the adaptor. In alternative embodiments, the deaminated products is not amplified and, instead, it is sequenced directly (e.g., by nanopore or PacBio sequencing).

In some embodiments, the method may comprise enriching for target molecules using a probe that is complementary to a sequence in the original double-stranded fragment of DNA. This enrichment step could occur after deamination and in some cases may be done after the amplification step. In this step, the probe may be biotinylated and, in some embodiments, the deaminated products or amplification products may be hybridized with one of more probes. The target products can then be enriched by binding to a support (e.g., streptavidin beads).

In any embodiment, the method may further comprise sequencing the deaminated product, or an amplification product thereof, to produce sequence reads. This may be done using any suitable system including Illumina's reversible terminator method (see, e.g., Shendure et al, Science 2005 309:1728). The sequencing step may result in at least 10,000, at least 100,000, at least 500,000, at least 1M at least 10M at least 100M, at least 1 B or at least 10 B sequence reads per reaction. In some cases, the reads may be paired-end reads, thereby allowing both strands of the original molecule to be analyzed.

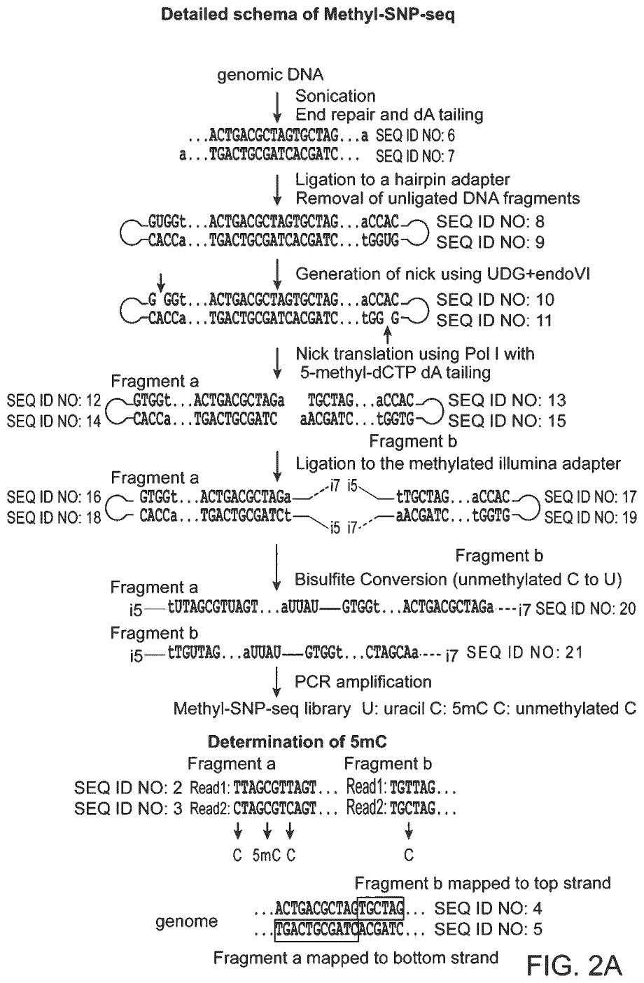

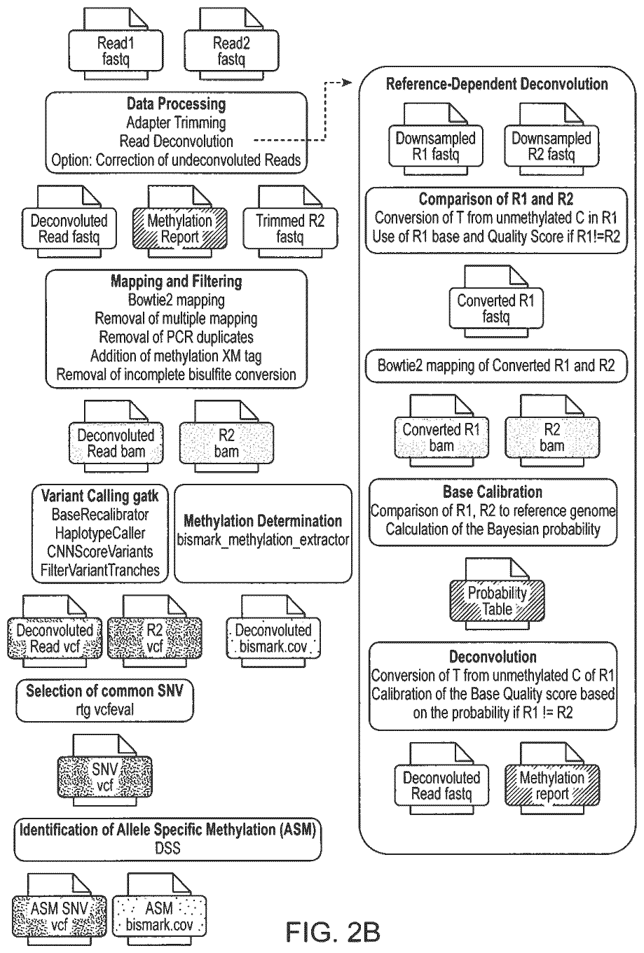

FIG. 1B illustrates how modified cytosines in the original strand can be identified. In this example, the paired end reads (i.e., Read1 and Read2) can be directly compared. As illustrated, T's in a Read1 sequence that correspond to a C in the Read2 sequence correspond to a C in the original strand, and C's in a Read1 sequence that correspond to a C in the Read2 correspond to a modified (methylated) C in the original strand. As such, in some embodiments, the method may comprise identifying a C in the sequence corresponding to the original strand, wherein the identified C corresponds to a modified nucleotide in the double-stranded fragment of DNA. FIG. 2B illustrates some of the data processing steps that could be employed to analyze the sequence reads. A modified C can be mapped to a site in a reference genome in some embodiments. That site may be annotated as being modified in the sample.

In any embodiment, the double-stranded fragment of DNA may be a fragment of eukaryotic, e.g., mammalian DNA, although in many cases the DNA can be from any source. The DNA in the initial sample may be made by extracting genomic DNA from a biological sample, and then fragmenting it. In some embodiments, the fragmenting may be done mechanically (e.g., by sonication, nebulization, or shearing) or using a double stranded DNA “dsDNA” fragmentase enzyme (New England Biolabs, Ipswich MA). In some embodiments, after the DNA is fragmented, the ends are polished and A-tailed prior to ligation to the adaptor. In other embodiments, the DNA in the initial sample may already be fragmented (e.g., as is the case for FPET samples and circulating cell-free DNA (cfDNA)). In any embodiment, fragments in the initial sample may have a median size that is below 1 kb (e.g., in the range of 50 bp to 500 bp, or 80 bp to 400 bp), although fragments having a median size outside of this range may be used.

One implementation of the method is illustrated in FIG. 2A. In this implementation, both ends of the double-stranded fragment of DNA are ligated to the hairpin adaptor and, as illustrated, the top and bottom strands of the double-stranded fragment of DNA become separated during the nick translation step. In this embodiment, the fragments are generated by sonicating genomic DNA and then repairing the ends and A-tailing the fragments. In this embodiment, there is a “U” in the 3′ stem of the hairpin adaptor, which is cleaved using USER (which is a mixture of UDG and endoVI), which leaves a 3′ hydroxyl that can be extended by a strand-displacing or nick-translating polymerase. The nick can also be produced by an endonuclease, a nicking endonuclease or an RNase, for example. In this example, the nick translation step is done by DNA polymerase I, although any nick-translating polymerase could be used. In other embodiments, a strand-displacing polymerase (e.g., a phi29 or Bst polymerase such as Bst2.0, for example) could be used with a similar result.

In some embodiments, the Methyl-SNP-seq method could alternatively be performed using duplex sequencing (see Schmitt et al Proc. Natl. Acad. Sci. 2012 109: 14508-14513). In these embodiments, the adaptor is a double-stranded adaptor without the hairpin, where the strands have complementary index sequences. The strands are sequenced separately in this alternative embodiment. However, the sequence reads can be grouped by the index sequence.

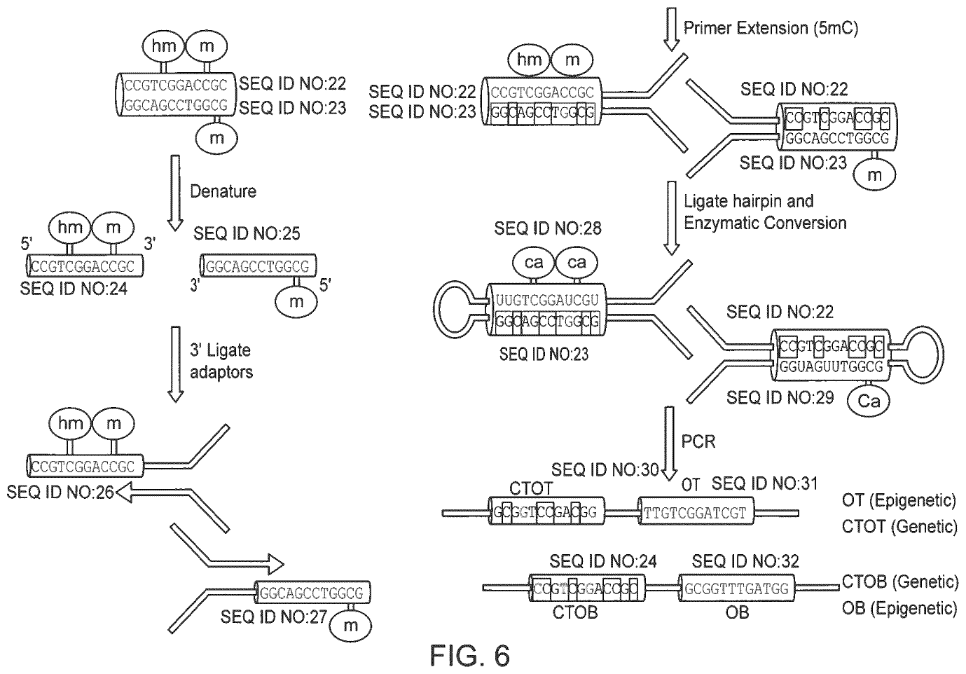

An alternative implementation is illustrated in FIG. 6, in which the double-stranded fragment of DNA is ligated to a hairpin adaptor and a double-stranded adaptor.

Also provided is a reaction mix comprising (a) a hairpin DNA that has a free 3′ end in a double stranded region of the hairpin DNA, (b) a strand-displacing or nick-translating polymerase, and (c) dGTP, dATP, dTTP, modified dCTP and no dCTP. In these embodiments, the hairpin DNA may comprise a fragment of mammalian DNA (e.g., a molecule of cfDNA) ligated to a hairpin adaptor. In these embodiments, the modified dCTP may be dmCTP, pyrrolo-dCTP or N⁴-dmCTP, for example.

Also provided are a variety of reaction intermediates, for example a nucleic acid molecule comprising, in order from 5′ to 3′: a first sequence, a linker, and a second sequence, wherein: the first sequence (which may be 50-500 nt in length) is composed of Gs, As, Ts, Cs and modified Cs; the second sequence (which may be 50-500 nt in length) is composed of Gs, As, Ts, modified Cs and no Cs; and the first and second sequences are complementary. In another example, the nucleic acid molecule may comprise, in order from 5′ to 3′: a first sequence, a linker, and a second sequence, wherein: the first sequence (which may be 50-500 nt in length) is composed of Gs, As, Ts, Us and modified Cs and the second sequence (which may be 50-500 nt in length) is composed of Gs, As, Ts, modified Cs and no Cs; and the first and second sequences are complementary except for the Us in the first sequence. In either of these embodiments, the linker may be composed of Gs, As, Ts and modified Cs. Other reaction intermediates are exemplified in the schematics of the Figures (which in some instances depict specific examples of DNA sample sequences for illustrative purposes only).

Kits for performing methods described are also provided. A kit may contain any of the components described above, typical in separate containers. For example, a kit may comprise (a) a hairpin adaptor containing a U in a double-stranded region of the adaptor; (b) one or more enzymes that create a nick at the site of the U (e.g., USER or the like); (c) a modified dCTP; and (d) a nick-translating or strand-displacing polymerase. In some embodiments, the modified dCTP may be dmCTP, pyrrolo-dCTP or N⁴-dmCTP. In these embodiments, the adaptor may contain modified Cs and no Cs, e.g., mCTP, pyrrolo-CTP or N⁴-mCTP. In some embodiments, the kit may further comprise a deaminase, wherein the modified Cs in the adaptor and modified dCTP are deamination resistant. In another embodiment, for example, as described in Example 10, a kit may comprise one or more of: (a) a double stranded adaptor; (b) a hairpin adaptor; (c) a modified dCTP and (d) a nick-translating or strand-displacing polymerase.

Other aspects of the methods include the following:

When dsDNA is the product of fragmentation of a genome; (a) the method may further comprise ligating a linker to both ends of the dsDNA; the linker is a loop adaptor having a double-stranded stem sequence for ligating to the dsDNA wherein the stem sequence contains a nick site; the linker is a chemical linkage group; the nick site is an uracil and nicking occurs by means of endonuclease III, endonuclease V or Fpg and uracil deglycosylase; the nick site is inosine and the nicking occurs by means of endonuclease V; the nick site is a restriction endonuclease recognition sequence and nicking occurs by means of a nicking endonuclease; the nick site is a ribonucleotide and nicking occurs by means of an RNAse; the nick site is 8-oxo-G and nicking occurs by means of Fpg; the unprotected base is cytosine and (c) further comprises converting the unprotected base with sodium bisulfite wherein cytosine is converted to thymine; the unprotected base is cytosine and (c) further comprises converting the unprotected base with a methyl dioxygenase and a deaminase so that cytosine is converted to thymine; the unprotected base is methylcytosine and further comprises converting the unprotected base with reducing boron and a methyl dioxygenase so that methylcytosine is converted to thymine; amplifying the ssDNA; exponential or linear amplification; sequencing amplicons to obtain read 1 and read 2 or wherein amplification is optional for sequencing using nanopores; deconvoluting read 1 and read 2 to identify the location and/or mapping of the modified bases; and/or deconvoluting using a computer system, comprising a computer and a program.

A composition may include a ssDNA having a first portion and a second portion wherein the first portion and the second portion are linked through an intermediate portion; wherein (a) the first portion has a naturally occurring sequence comprising no modified cytosine or one or more modified cytosines; (b) the second portion has a sequence that is complementary to the first portion but where either every cytosine or every modified cytosine in the sequence is artificially replaced by a protected nucleotide; and (c) the intermediate portion linking the first portion to the second portion is an artificial nucleic acid sequence or other chemical composition.

Additional features of the composition may include one or more of the following: The modified cytosine is methylated cytosine and/or hydroxymethylcytosine; the protected nucleotide is distinguishable by sequencing from an unprotected nucleotide; and/or the protected nucleotide is recorded as cytosine in a sequencing read and the unprotected nucleotide is recorded as an altered base such as thymine in a sequencing read.

In general, a composition is provided that includes: (a) a double-stranded fragment having a first strand with a 5′ end and a second complementary strand with a 3′ end opposite to the 5′ end; and (b) a linker between the 5′ end of the first strand and the 3′ end of the second strand.

In any of the compositions or methods described above, the linker may contain a degenerate sequence to uniquely identify the dsDNA.

### Embodiments

Embodiment 1. A method for determining the presence of, and/or mapping modified cytosines in double-stranded DNA (dsDNA) fragments, comprising:
(a) ligating a linker to an end of the dsDNA:
(b) nicking the linker at or proximate to a 5′ end of the dsDNA to permit strand displacement copying of a template strand in the dsDNA to form a neosynthesized strand, in the presence of:
i. a modified dCTP and unmodified dATP, dTTP and dGTP so that the modified cytosine in the neosynthesized strand is protected and unmodified cytosine in the template strand is unprotected; or
ii. an unmodified cytosine and unmodified dATP, dTTP and dGTP so that the unmodified cytosine in the neosynthesized strand is protected and any modified cytosine in the template strand is unprotected;
(c) converting the unprotected base to another base, and causing the dsDNA to become linearized to form a single-stranded DNA (ssDNA), having a first portion corresponding to the template strand, a central portion and a second portion corresponding to the neosynthesized strand;
(d) providing a first read of the template strand and a second read of the neosynthesized strand; and
(e) comparing the first read to the second read to determine the presence of, and/or map the modified nucleotides in the dsDNA.

Embodiment 2. The method according to embodiment 1, wherein the dsDNA is the product of fragmentation of a genome.

Embodiment 3. The method according to embodiment 1 or 2, wherein (a) further comprises ligating a linker to both ends of the dsDNA.

Embodiment 4. The method according to any previous embodiment, wherein the linker is a loop adaptor having a double-stranded stem sequence for ligating to the dsDNA wherein the stem sequence contains a nick site.

Embodiment 5. The method according to any of embodiments 1-3, wherein the linker is a chemical linkage group.

Embodiment 6. The method according to any previous embodiment, wherein the nick site is an uracil and nicking occurs by means of endonuclease III, endonuclease V or Fpg and uracil deglycosylase.

Embodiment 7. The method according to any of embodiments 1-5, wherein the nick site is inosine and the nicking occurs by means of endonuclease V.

Embodiment 8. The method according to any of embodiments 1-5, wherein the nick site is a restriction endonuclease recognition sequence and nicking occurs by means of a nicking endonuclease.

Embodiment 9. The method in any of embodiments 1-5 wherein the nick site is a ribonucleotide and nicking occurs by means of an RNAse.

Embodiment 10. The method in any of embodiments 1-5, wherein the nick site is 8-oxo-G and nicking occurs by means of Fpg.

Embodiment 11. The method according to any of the previous embodiments, wherein the unprotected base is cytosine and (c) further comprises converting the unprotected base with sodium bisulfite wherein cytosine is converted to thymine.

Embodiment 12. The method according to any of embodiments 1-10, wherein the unprotected base is cytosine and (c) further comprises converting the unprotected base with a methyl dioxygenase and a deaminase so that cytosine is converted to thymine.

Embodiment 13. The method according to any of embodiments 1-10, wherein the unprotected base is methylcytosine and (c) further comprises converting the unprotected base with reducing boron and a methyl dioxygenase so that methylcytosine is converted to thymine.

Embodiment 14. The method according to any of the previous embodiments, wherein (c) further comprises amplifying the single-stranded DNA.

Embodiment 15. The method of embodiment 14, wherein amplifying is exponential.

Embodiment 16. The method of embodiment 14, wherein amplifying is linear.

Embodiment 17. The method according to any previous embodiment, wherein (e) further comprises sequencing amplicons to obtain Read 1 and Read 2, or wherein amplification is optional for sequencing using nanopores.

Embodiment 18. The method according to embodiment 17, further comprising deconvoluting Read 1 and Read 2 to identify the location and/or mapping of the modified bases.

Embodiment 19. The method according to embodiment 18, wherein the deconvoluting is performed by a computer system, comprising a computer and a program.

Embodiment 20. A composition comprising a single-stranded DNA (ssDNA) having a first portion and a second portion wherein the first portion and the second portion are linked through an intermediate portion; wherein:
(a) the first portion has a naturally occurring sequence comprising no modified cytosine or one or more modified cytosines;
(b) the second portion has a sequence that is complementary to the first portion but where either every cytosine or every modified cytosine in the sequence is artificially replaced by a protected nucleotide; and
(c) the intermediate portion linking the first portion to the second portion is an artificial nucleic acid sequence or other chemical composition.

Embodiment 21. The composition according to embodiment 20, wherein the modified cytosine is methylated cytosine and/or hydroxymethylcytosine.

Embodiment 22. The composition according to embodiment 20, wherein the protected nucleotide is distinguishable by sequencing from an unprotected nucleotide.

Embodiment 23. The composition according to embodiment 22, wherein the protected nucleotide is recorded as cytosine in a sequencing read and the unprotected nucleotide is recorded as an altered base such as thymine in a sequencing read.

Embodiment 24. A composition, comprising: (a) a double-stranded fragment having a first strand with a 5′ end and a second complementary strand with a 3′ end opposite to the 5′ end; and (b) a linker between the 5′ end of the first strand and the 3′ end of the second strand.

Embodiment 25. The composition according to any of embodiments 20-24, wherein the linker contains a degenerate sequence to uniquely identify the dsDNA.

Embodiment 26. A method for generating a deamination-resistant strand of DNA, comprising:
(a) ligating a hairpin adaptor to a double-stranded fragment of DNA to produce a ligation product;
(b) enzymatically generating a free 3′ end in a double-stranded region of the hairpin adaptor in the ligation products; and
(c) extending the free 3′ end in a dCTP-free reaction mix that comprises a strand-displacing or nick-translating polymerase, dGTP, dATP, dTTP and modified dCTP to generate a hairpin product that has an original strand and a neosynthesized strand that contains modified Cs.

Embodiment 27. The method of Embodiment 26, further comprising
(d) deaminating the hairpin product or an adaptor-ligated product thereof, wherein the modified Cs protect the neosynthesized strand from deamination.

Embodiment 28. The method of Embodiment 27, wherein the deaminating is done using bisulfite.

Embodiment 29. The method of Embodiment 27, wherein the deaminating is done using a cytosine deaminase, optionally after enzymatically protecting any modified Cs in the original strand from deamination.

Embodiment 30. The method of Embodiment 29, wherein the cytosine deaminase modifies a double-stranded or single-stranded substrate.

Embodiment 31. The method of any of Embodiments 27-30, further comprising amplifying the deaminated product of step (d) thereby converting any deaminated Cs to Ts in the amplification product.

Embodiment 32. The method of Embodiment 31, further comprising enriching for target molecules using a probe that is complementary to a sequence in the double-stranded fragment of (a).

Embodiment 33. The method of any of Embodiments 27-32, further comprising sequencing the deaminated product, or an amplification product thereof, to produce sequence.

Embodiment 34. The method of Embodiment 33, further comprising identifying a C in the sequence corresponding to the original strand, wherein the C corresponds to a modified cytosine. Embodiment 35. The method of Embodiment 34, further comprising mapping the modified cytosine to a site in a reference genome and annotating the site as being modified. Embodiment 36. The method of any prior Embodiment, wherein the modified dCTP is dmCTP, pyrrolo-dCTP or N⁴-dmCTP.

Embodiment 37. The method of any prior Embodiment, wherein the double-stranded fragment of DNA is a fragment of mammalian DNA.

Embodiment 38. The method of any prior Embodiment, wherein the double-stranded fragment is a molecule of cfDNA.

Embodiment 39. The method of any prior Embodiment, further comprising enzymatically modifying the double-stranded fragment of DNA, the ligation product or hairpin product to protect any modified cytosines or hydroxymethylcytosines from deamination.

Embodiment 40. The method of any prior Embodiment, wherein in step (a) both ends of the double-stranded fragment of DNA are ligated to the hairpin adaptor and in step (b) the top and bottom strands of the double-stranded fragment of DNA become separated.

Embodiment 41. The method of any prior Embodiment, wherein step (b) is done using USER, an endonuclease, a nicking endonuclease or an RNase.

Embodiment 42. The method of any prior Embodiment, wherein the hairpin adaptor has at least one modified C and no Cs.

Embodiment 43. The method of any prior Embodiment, wherein the modified C of the adaptor is mCTP, pyrrolo-CTP or N⁴-mCTP.

Embodiment 44. A reaction mix comprising:
(a) a hairpin DNA that has a free 3′ end in a double-stranded region;
(b) a strand-displacing or nick-translating polymerase, and
(c) dGTP, dATP, dTTP, modified dCTP and no dCTP.

Embodiment 45. The reaction mix of Embodiment 44, wherein the hairpin DNA comprises a fragment of mammalian DNA ligated to a hairpin adaptor.

Embodiment 46. The reaction mix of Embodiment 44, wherein the hairpin DNA comprises a molecule of cfDNA ligated to a hairpin adaptor.

Embodiment 47. The reaction mix of any of Embodiment 44-46, wherein the modified dCTP is dmCTP, pyrrolo-dCTP or N⁴-dmCTP.

Embodiment 48. A nucleic acid molecule comprising, in order from 5′ to 3′: a first sequence, a linker, and a second sequence, wherein: the first sequence is composed of Gs, As, Ts, Cs and modified Cs; the second sequence is composed of Gs, As, Ts, modified Cs and no Cs; and the first and second sequences are complementary.

Embodiment 49. A nucleic acid molecule comprising, in order from 5′ to 3′: a first sequence, a linker, and a second sequence, wherein: the first sequence is composed of Gs, As, Ts, Us and modified Cs and the second sequence is composed of Gs, As, Ts, modified Cs and no Cs; and the first and second sequences are complementary except for the Us in the first sequence.

Embodiment 50. A kit for generating a deamination-resistant strand of DNA, comprising:
(a) a hairpin adaptor containing a U in a double-stranded region of the adaptor;
(b) one or more enzymes that create a nick at the site of the U;
(c) a modified dCTP; and
(d) a nick-translating or strand-displacing polymerase.

Embodiment 51. The kit of Embodiment 50, wherein the modified dCTP is dmCTP, pyrrolo-dCTP or N⁴-dmCTP.

Embodiment 52. The kit of Embodiment 50 or 51, wherein the adaptor contains modified Cs and no Cs.

Embodiment 53. The kit of Embodiment 52, wherein the modified Cs of the adaptor are mCTP, pyrrolo-CTP or N⁴-mCTP.

Embodiment 54. The kit of any of Embodiments 50-53, further comprising a deaminase, wherein the modified Cs are deamination resistant.

Embodiment 55. A method for generating a deamination-resistant strand of DNA, comprising: (a) separating the strands of a double-stranded fragment of DNA to produce a single-stranded fragment; (b) attaching a double-stranded adaptor to the 3′ end of the single-stranded fragment;
(c) extending the free 3′ end of an attached double-stranded adaptor in a dCTP-free reaction mix that comprises a strand-displacing or nick-translating polymerase; and dGTP, dATP, dTTP, and modified dCTP, to generate a double-stranded product; (d) attaching a hairpin adaptor to the 5′ end of the double-stranded product to generate a hairpin product that has an original strand and a neosynthesized strand that contains modified Cs.

Embodiment 56. The method of Embodiment 55, further comprising deaminating the hairpin product to produce a deaminated hairpin product, wherein the modified Cs protect the neosynthesized strand from deamination.

Embodiment 57. The method of Embodiment 56, wherein the deaminating is done using bisulfite.

Embodiment 58. The method of Embodiment 56, wherein the deaminating is done using a cytosine deaminase.

Embodiment 59. The method of Embodiment 56, wherein prior to deaminating, any modified Cs are enzymatically protected from deamination.

Embodiment 60. The method of Embodiment 55, wherein the double-stranded adaptor further comprises a unique molecular identifier.

Embodiment 61. The method of Embodiment 60, wherein the unique molecular identifier is a known sequence.

Embodiment 62. The method of Embodiment 60, wherein the unique molecular identifier is a random sequence.

Embodiment 63. The method of Embodiment 55, wherein the hairpin adaptor is attached by ligation.

Embodiment 64. The method of Embodiment 63, wherein the hairpin adaptor is attached by ligating a linear double-stranded DNA to the double-stranded product and circularizing the linear double-stranded DNA to produce the hairpin adaptor.

Embodiment 65. The method of Embodiment 56, further comprising amplifying the deaminated hairpin product to produce an amplified product.

Embodiment 66. The method of any Embodiment of Embodiment 55, further comprising sequencing the deaminated hairpin product or the amplified product, to produce sequence.

Embodiment 67. The method of Embodiment 65, further comprising enriching for target molecules using a probe that is complementary to a sequence in the double-stranded fragment of (a).

Embodiment 68. The method of Embodiment 66, further comprising identifying a C in the sequence corresponding to the original strand, wherein the C corresponds to a modified cytosine.

Embodiment 69. The method of Embodiment 68, further comprising mapping the modified cytosine to a site in the reference genome and annotating the site as being modified.

Embodiment 70. The method of any Embodiment of Embodiment 55, wherein the modified dCTP is dmCTP, pyrrolo-dCTP or N⁴-dmCTP.

Embodiment 71. The method of any Embodiment of Embodiment 55, wherein the double-stranded fragment of DNA is a fragment of mammalian DNA.

Embodiment 72. The method of any Embodiment of Embodiment 55, wherein the double-stranded fragment is a molecule of cfDNA.

Embodiment 73. The method of any Embodiment of Embodiment 55, wherein the hairpin adaptor has at least one modified C and no Cs.

Embodiment 74. The method of Embodiment 73, wherein the modified C of the adaptor is mCTP, pyrrolo-CTP or N⁴-mCTP.

Embodiment 75. A kit for generating a deamination-resistant strand of DNA in accordance with the method of Embodiment 55.

Embodiment 76. A reaction mix for generating a deamination-resistant strand of DNA in accordance with the method of Embodiment 55.

### EXAMPLES

The following examples are put forth so as to provide those of ordinary skill in the art with additional disclosure and description of how to make and use the present invention, and are not intended to limit the scope of what the inventors regard as their invention nor are they intended to represent that the experiments below are all or the only experiments performed.

### Example 1: Principles of Methyl-SNP-Seq

Methyl-SNP-seq takes advantage of the double stranded nature of DNA to duplicate the sequence information into a linked copy to the original strand that is resistant to bisulfite conversion. After conversion, the copied strand conserves its original four nucleotide content while the original strand undergoes deamination at un-methylated cytosines. Both strands are sequenced using Illumina paired-end sequencing resulting in one read containing the sequence information while the other paired-read containing the methylation information (FIGS. 1A and 2A).

To achieve this, a hairpin adaptor is ligated to the fragmented double stranded DNA, forming a dumbbell shaped DNA. Next, nick at both opposite ends of the adaptors are introduced and using nick translation, a copy of the original strand is synthesized, the other strand remains unchanged. To make this strand resistant to conversion, 5mCTP are replacing CTP as a source of nucleotide. This nick translation step broke the dumbbell shaped DNA somewhere in the middle of the fragment, creating a blunt end. Methylated Illumina Y-shaped adaptors are ligated to the blunt-ends before bisulfite conversion. Conversion opened the closed DNA structure revealing a single strand DNA molecule that can be amplified using the Illumina adaptors. Sequencing requires paired-end reads to obtain both the methylation and the genomic sequence information.

The protocol was designed so that the Read1 of the paired-end read pair provides the bisulfite conversion information while the corresponding Read2 provides the genome sequence. To combine both information together, we developed a deconvolution algorithm (FIGS. 1B and 2B) that compares Read1 with Read2 considering the conversion and complementary nature of the paired-end reads. This step, called the read deconvolution step, accurately identifies each cytosine and its methylation status. More specifically, a T in Read1 pairing with a C in Read2 corresponds to an unmethylated C, while a C in Read1 pairing with a C in Read2 corresponds to a methylated C (FIG. 1B). All remaining pairs should follow the canonical base pairing of double stranded DNA.

A typical Methyl-SNP-seq experiment yields about 85-90% of the reads being deconvoluted. Within the deconvoluted reads, around 98-99% of the positions show either a direct agreement between pairs or a profile consistent with cytosine conversion. The remaining 1-2% of bases that disagreed may be resulting from damages caused by the bisulfite reaction or errors generated during nick translation, PCR amplification or sequencing. In this case, we cannot differentiate the correct base. Accordingly, we use the Read1 base as the deconvoluted base but adjust the Phred quality score to mark this disagreement as a potential error. The adjustment of the Phred quality scores in case of a pair disagreement depends on whether a reference genome is available or not. If a reference genome is available (Reference-dependent Read Deconvolution), base calibration is performed using Bayesian statistics which considers the corresponding nucleotide on the reference genome, the substitution type and the position on the read. Thus, the adjusted Phred quality score reflects the Bayesian probability that the Read1 base is true. If a reference genome is unavailable (Reference-free Read Deconvolution), the Phred quality score is assigned to 0.

The deconvolution step results in a fastq file that contains deconvoluted reads with adjusted Phred quality scores and, for each cytosine, its methylation status in a methylation report file. The pipeline for processing and deconvoluting the linked paired-end reads is freely available in Github (link). The output of the deconvolution pipeline is in a standard format compatible with existing algorithms designed for genome assembly, genetic variant calling (e.g. GATK (McKenna et al. 2010)) and methylation quantification (e.g. Bismark (Krueger and Andrews 2011)). The ability to distinguish between a methylated and unmethylated cytosine directly on the unmapped read while simultaneously obtaining the original genomic sequences is the key strength of this technology.

Short read high throughput sequencing technologies typically erase all information about DNA modifications and only retain the 4 canonical base arrangement. The analysis of epigenetic phenomenon is usually performed using specialized technologies. To capture epigenetic information on conventional high throughput sequencers, the following method (referred to as “Methyl-SNP-seq”) was developed. The technology that takes advantage of the redundancy of the double helix, to extract the methylation and sequence information from a single original DNA molecule. More specifically, Methyl-SNP-seq involves deaminating (e.g., enzymatically or by bisulfite conversion) one of the double strands to identify methylation while the other strand is left intact for sequencing. Both strands are locked together to link the dual readout on a single paired-end read. Because one of the strands retains the original 4 nucleotide composition, Methyl-SNP-seq can be used in conjunction with sequence specific probes for targeted enrichment or amplifications. We demonstrate the usefulness of this technology on a broad spectrum of applications ranging from allele specific methylation analysis in humans to methylation identification in complex bacterial communities. Amplification based sequencing methods provide only the sequential arrangement of the canonical four bases A, T C and G while all modifications, originally present on the DNA, are erased. The information on what base was originally modified is lost during the in-vitro DNA synthesis steps that happen during amplification, clustering, and sequencing. To circumvent this limitation and obtain cytosine methylation information, techniques such as bisulfite sequencing convert unmethylated Cs to Ts before subjecting the converted DNA to sequencing. A T output after bisulfite treatment is therefore ambiguous: it corresponds to either a naturally occurring T in the sequence or a deaminated unmodified C and a reference genome is therefore required to distinguish the two possibilities. This ambiguity is the major drawback in bisulfite sequencing and relegate all the techniques that rely on deamination to applications directed for methylation analysis only.

By bridging the double strand together, Methyl-SNP-seq takes advantage of the redundant information captured in the complementing strands to obtain both the arrangement of the canonical four bases and the methylation information. The accuracy of the dual readouts of Methyl-SNP-seq is comparable to state-of-the-art techniques for both SNPs and methylation analysis. Because the sequencing power is allocated to a dual readout, the sensitivity for each single readout is reduced to effectively a single-end read instead of a paired-end read. This affects notably the ability to perform assemblies as most of the assemblers have been optimized for paired-end sequencing. With the ability to read longer stretches of sequence, this limitation can be partially overcome. Furthermore, there should be no technical limitations from the manufacturer in adapting the instrument to perform dual paired end read sequencing using the invariable loop sequence as primer.

The efficiency of Methyl-SNP-seq is much higher than performing the WGBS and DNA-seq separately. In addition Methyl-SNP-seq offers important functionalities that are not feasible when performing WGBS or DNA-seq. Notably, Methyl-SNP-seq leaves one of the double strands intact by incorporating m5CTP instead of CTP in the neo-synthesized fragment. This is conceptually a significant improvement compared to another method in which both strands are subjected to deamination. In the latter case, the ability to obtain the original sequence can only be done computationally, by aligning and deconvoluting paired end reads. By keeping intact the 4-nucleotide-based strand, Methyl-SNP-seq is compatible with conventional probe sets for target enrichment. Indeed, we show similar on-target performance for both conventional DNA-seq and Methyl-SNP-seq exome sequencing.

Retaining the original sequence is also useful for any target-specific amplification such as CRISPR-based targeting, and other sequence specific technologies. Beyond sequence specific applications, we demonstrate the applicability of Methyl-SNP-seq in directly demonstrating allele specific methylation at single molecule resolution. In conjunction with target-specific amplification and sequencing, Methyl-SNP-seq is an ideal technique to validate candidate ASMs derived from Methylome-Wide Association Studies.

Beyond human methylomes, Methyl-SNP-seq is a useful technology notably for organisms for which a reference genome is not available such as non-model organisms and microbial communities. Notably, the identification of modification directly on the unmapped reads enhanced the ability to bin sequences based on methylation patterns, an important feature for resolving genomes within a complex community (Wilbanks et al. 2022) (Tourancheau et al. 2021). The ability to obtain the original genomic sequence allows further functionalities specific to organisms for which a reference genome is unavailable or variations between the studied organism and its reference genome is too high to confidently distinguish methylation from transition SNPs. For example, we demonstrate the ability to perform assemblies and overlay methylation on the newly assembled genome.

### Example 2: Preparation of Sequencing Libraries and Sequencing

For human Methyl-SNP-seq sequencing, genomic DNA isolated from the GM12878 cell line (NA12878, provided by Coriell Institute) was used for library preparation. For human whole genome Methyl-SNP-seq sequencing, 4 ug of NA12878 gDNA was used and unmethylated lambda DNA was spiked in to monitor bisulfite conversion efficiency. The genomic DNA was fragmented using 250bp sonication protocol using a Covaris S2 sonicator. Two technical replicates were set up. For human exome Methyl-SNP-seq sequencing, 4 ug of NA12878 gDNA was fragmented using 400bp or 500bp sonication protocol.

For bacteria Methyl-SNP-seq sequencing, 2 ug of E. coli genomic DNA (MG1655 strain) or 2 ug of mixed bacterial DNA (containing 1 ug of E. coli MG1655 genomic DNA and 1 ug of C. acetobutylicum genomic DNA) was used. The genomic DNA was fragmented using 250bp sonication protocol. 100 ng of C. acetobutylicum genomic DNA was to prepare an EMseq library (NEB E7120) as directed by the manufacturer. The library was sequenced using an Illumina Nextseq 550 sequencer for 75 bp paired end reads.

As shown in FIG. 2A, the fragmented gDNA was end repaired and dA-tailed (NEB Ultra II E7546 module), then ligated to the custom hairpin adaptor using NEB ligase master mix (NEB, M0367). The incomplete ligation product (fragment having only one or no adaptor ligated) was removed using exonuclease (NEB exolll and NEB exoVII). Two nick sites were created at the Uracil positions in the hairpin adaptors at both ends after being treated with UDG and endoVIII. The nick sites were translated towards 3′ terminus by DNA polymerase I in the presence of dATP, dGTP, dTTP and 5-methyl-dCTP. The nick translation causes double stranded DNA break when DNA polymerase I encounters the other nick on the opposite strand. The resulting fragments have one end ligated to a hairpin adaptor and blunt end on the other side. The blunt end was dA-tailed and ligated with methylated Illumina adaptor. The ligated product was bisulfite converted using Abcam Fast Bisulfite conversion kit (Abcam, ab117127). The bisulfite converted product was amplified using NEBNext Q5U Master Mix (NEB, M0597). The resulting indexed library was used for Illumina sequencing or target enrichment.

To perform targeted sequencing, about 200 ng-300 ng Methyl-SNP-seq indexed library was used in a pool for target enrichment. The whole human exome regions were enriched from the pooled libraries using the Twist Human Core Exome panel (Twist, 102025) following the manufacturer's instructions. The enriched DNA fragments were further amplified using NEBNext Q5 Master Mix (NEB, M0544) and NEBNext Library Quant Primer Mix (NEB, E7603) for sequencing.

The human Methyl-SNP-seq libraries (WGS sequencing and targeted sequencing) were sequenced using an Illumina Novaseq 6000 sequencer for 100bp paired end reads. The bacteria Methyl-SNP-seq libraries (E. coli or mixed sample) were sequenced using an Illumina Nextseq 550 sequencer for 150bp paired end reads.

The sequence of the hairpin adaptor (46bp) sequence is shown below:

(SEQ ID NO: 1)
5′-(p)CCACGACGACGACGACGAGCGTTAGGCTCGTCGTCGTCGTCGU
GGT-3′

### Example 3: Analysis of Sequencing Data

Data Processing for Methyl-SNP-seq: The sequencing reads were trimmed for both Illumina adaptor and hairpin adaptor using Trimgalore version 0.6.4. For human NA12878 Methyl-SNP-seq sequencing, the bases of last cycle [cycle 100] for both Read1 and Read2 were further trimmed due to poor quality.

Next was Read Deconvolution, which determines the base, adjusts the base quality score and extracts the methylation information by comparing the paired Read1 and Read2. This step generates a fastq file containing the deconvoluted reads and a corresponding methylation report. The principle of Read Deconvolution is explained bellow (see also FIG. 2B).

Reference-free Read Deconvolution was performed using a custom pipeline that includes the following steps:
(1) Base determination and methylation extraction. For the same Illumina cycle, if Read1 base is a C and Read2 base is a C, it results in a C in the deconvoluted read and a 5mC in the methylation report; while if Read1 base is a T and Read2 base is a C, it results in a C in the deconvoluted read and a unmethylated C in the methylation report.
(2) Base quality score adjustment. For the mismatching positions that Read1 bases are different from Read2 bases except for the Read1-T Read2-C case, Read1 bases are used but the sequencing quality scores are adjusted to 0 in the deconvoluted reads.

Reference-dependent Read Deconvolution was done using the following steps:
(1) Base determination and methylation extraction is the same as the Reference-free Read Deconvolution. But Reference-dependent Read Deconvolution uses a statistical model for the base quality score adjustment as shown below.
(2) Base quality score adjustment. For the mismatching positions, by comparing to the reference genome, a Bayesian probability is calculated, which reflects the likelihood of being able to trust the Read1 base. Therefore, Read1 bases are used but the sequencing quality scores are adjusted based on the Bayesian probability in the deconvoluted reads.

Alignment and Data Filtering for human NA12878 Methyl-SNP-seq (FIG. 2A): For human NA12878 Methyl-SNP-seq, the Deconvoluted Reads were aligned to the GRCh38 human reference genome using bowtie2 (version 2.3.0) default parameter for single end mapping with the addition of read group identifier defined by —rg-id and —rg. These identifiers including the information for sequencing platform, flow cell and lane, barcode and sample were necessary for Base Quality Score Recalibration by gatk for variant calling.

To achieve high accuracy, the following steps were taken to filter the aligned data before variant calling and methylation status determination: (1) removal of multiple mapping using an inhouse script. Here for bowtie2 single end mapping, the unique mapping is defined as the read having only AS tag or AS score !=XS score (bowtie2 AS: best alignment score, XS: second best alignment score); (2) removal of PCR duplicates using an inhouse script. Here for bowtie2 single end mapping, the PCR duplicates are identified as reads aligned to the same position as well as having the same sequence; (3) addition of XM tag reflecting the methylation status. Based on the Methylation report generated in the Read Deconvolution step, a XM tag is added to each mapped read in sam file using an inhouse script. The XM tag is defined by bismark to mark methylation call string and used to extract methylation status; (4) removal of reads having incomplete bisulfite conversion using bismark (version 0.22.3) filter non conversion.

The resulting filtered Deconvoluted Reads from two replicates were combined to be used for variant calling and methylation determination. There were 1.6 billion and 11 million filtered deconvoluted reads for human WGS and exome targeted Methyl-SNP-seq, respectively.

Data Processing for human NA12878 whole genome sequencing: Whole genome sequencing of human NA12878 generated by JIMB NIST Genome in a Bottle (Zook et al. 2016) (JIMB WGS HG001) was used as a benchmark for comparison with Methyl-SNP-seq for variant calling. For a fair comparison to avoid differences due to the choice of variant calling pipeline (Cornish and Guda 2015), we processed the JIMB WGS data set using the same strategy as for the human Methyl-SNP-seq: (1) shortening the paired end reads to 99bp; (2) trimming Illumina adaptor; (3) bowtie2 mapping for the paired-end reads; (4) removing multiple alignments and PCR duplicates using samtools (version 1.14) markdup; (5) removing multiple mapping using the inhouse script (https://github.com/elitaone/Methyl-SNP-seq/ReadProcessing/MarkUniread.py). To achieve a similar coverage, we downsampled to use 1.6 billion filtered JIMB WGS reads for variant calling.

Data Processing for human NA12878 whole genome bisulfite sequencing: Whole genome bisulfite sequencing (WGBS) of human NA12878 generated by ENCODE (ENCSR890UQO) was compared with Methyl-SNP-seq for methylation quantification.

We shortened the paired end WGBS data to 99bp and trimmed the Illumina adaptors. The adaptor trimmed read pairs were aligned to the human GRCh38 genome using bismark (version 0.22.3). The properly paired reads were further filtered before methylation determination: (1) removing PCR duplicates using samtools markdup; (2) filtering out alignments having incomplete bisulfite conversion using bismark filter_non_conversion. The two ENCODE replicates were combined having about 1.6 billion filtered reads for methylation quantification.

Variant calling and SNV comparison: We performed variant calling on the filtered data set as mentioned above using gatk (version 4.1.8.1) following gatk best practice recommendations for germline short variant discovery. First, BaseCalibration (BaseRecalibrator and ApplyBQSR) was applied on the filtered data set to calibrate the systematic errors made by sequencing. Next, the calibrated reads were used for variant calling using HaplotypeCaller. Finally, FilterVariantTranches was applied to filter raw SNVs using—info-key CNN_1D and—snp-tranche 99—indel-tranche 99. For human targeted Methyl-SNP-seq sequencing, an additional filter ‘DP<6’ was applied to remove SNPs with low coverage. In this study, only SNVs on the somatic chromosomes, chrX and chrM were reported and used for analysis.

The common SNVs identified by both Deconvoluted Read and Read2 were used as the Methyl-SNP-seq defined genetic variants. We used vcfeval from RTG Tools (version 3.11) (Cleary et al. 2014) to compare the SNVs defined by Methyl-SNP-seq or the benchmark JIMB WGS.

Methylation quantification: For Methyl-SNP-seq and WGBS, the methylation information was extracted on the filtered reads or read pairs using bismark_methylation_extractor (version 0.22.3) with the following parameters: —single-end—merge_non_CpG—bedGraph. We also used the latest Nanopore sequencing data set of human GM12878 cell line for methylation detection (Jain et al. 2018). The Nanopore reads (in total 8.7 million from 21 runs) were aligned to the human GRCh38 genome using minimap2 (version 2.17). The methylation modification was detected using nanopolish (version 0.13.2) call-methylation function.

The methylation level of UCSC annotated CpG islands (CGI) was defined as: CGI methylation=number of methylated CpG Cs in the region/number of CpG Cs in the region Only the CGIs having coverage (number of CpG Cs in the region) above 50 were used for comparison between different methods.

Allele specific methylation determination: To discover the allele specific methylation loci in the NA12878 genome, we used the heterozygous SNPs detected by Methyl-SNP-seq and confirmed in the JIMB NA12878 SNP vcf file (Zook et al. 2019). We split the Methyl-SNP-seq reads into two groups based on the defined SNP: REF (reads having the reference SNP) and ALT (reads having the alternative SNP). The methylation status of CpG sites was extracted for each group using bismark_methylation_extractor as previously mentioned. Finally the differentially methylated region between REF and ALT group were detected using DSS tool (version 2.38.0) (Feng, Conneely, and Wu 2014) with the following threshold for callDML and callDMR function: delta=0.1, p.threshold=0.05.

Genome assembly of Methyl-SNP-seq of E. coli and mixed sample: Bacterial genomes were assembled using velvet (version 1.2.10) based on 16.4 million and 36.7 million deconvoluted reads for E. coli and mixed sample, respectively. The following parameters were used for velvet assembly to obtain the best result: for E. coli, k=81-fastq-short-exp_cov 13-cov_cutoff 9-min_contig_Igth 500; for mixed sample, k=75-fastq-short-exp_cov 15-cov_cutoff 8-min_contig_Igth 500. The assembly quality was estimated using QUAST (web interface) (Gurevich et al. 2013).

Determination of methylase recognition site based on the deconvoluted reads We randomly chose 2% deconvoluted reads to identify the methylase recognition site in E. coli or mixed sample (0.28 million or 0.67 million reads, respectively). The 8mers including 3bp upstream and 4bp downstream of either a 5mC or unmethylated C were extracted for each read. The reads including more than one methylated C were excluded from this analysis. The numbers of 8mers containing either 5mC or 5mC and unmethylated cytosine were counted. We used Binomial statistics with Bonferroni Correction to determine the 8mer sequences that have significantly higher methylation level compared to the background. The pvalue is calculated using the following formula.

Pvalue (of each 8mer sequence)=1−binom.cdf(k, n, PO)

For each 8mer sequence, k is the number of 8mers having 5 mC; n is the number of 8mers having 5 mC and unmethylated cytosine; P0 is average methylation level. We used a custom script to perform this statistical analysis.

These significantly enriched 8mer sequences were further clustered to create the motif logo using a hierarchical linkage method based on the difference between each pair of sequences. The number of clusters (—number) can be decided based on cluster heatmap. Specifically, in this study we assigned the significantly enriched sequences into 2 clusters for E. coli and 3 clusters for the mixed sample (FIG. 6B).

Bacterial contamination: Contigs from the contaminated bacteria were obtained. In order to identify the m5C methylase in this organism, we run prokka on the assembled contigs using the—hmms parameter with Pfam (Pfam-A.hmm, version 35) as the search hmmer database. This annotation step resulted in the finding of a single ORF containing the C-5 cytosine-specific DNA methylase domain (PF00145) (PROKKA_03238 C-5 cytosine-specific DNA methylase).

### Example 4: Application of Methyl-SNP-seq to Whole Genome Sequencing of Human GM12878 Genomic DNA

As proof of concept, we tested Methyl-SNP-seq using gDNA from the widely studied human cell line GM12878 (lymphoblastoid cell line) for which a large number of sequencing and methylation datasets are publicly available. Methyl-SNP-seq libraries were constructed using 4 ug of genomic DNA spiked-in with unmethylated lambda DNA to monitor the bisulfite conversion efficiency. Experiments were performed in duplicates using the same source of starting material to monitor the reproducibility of the method. Whole genome sequencing was done using Illumina Nova-seq resulting in an average of 1.5 billion 100bp paired-end reads per replicates.

During the deconvolution step of Methyl-SNP-seq, an average of ˜84% of reads were successfully deconvoluted and more than 95% of the deconvoluted reads were mapped to the reference human genome using bowtie2 (Langmead and Salzberg 2012) with both replicates showing similar alignment metrics (data not shown). To obtain a set of high confidence genetic variants and accurate methylation quantification, we applied stringent data filters to remove multiple mapping reads, PCR duplicates and reads showing incomplete bisulfite conversion. About 64% of mapped reads remained after applying these filters. FIG. 2B shows the data analysis workflow used for this experiment.

### Example 5: Methyl-SNP-seq Accurately Detects Genetic Variation

We assessed the ability of Methyl-SNP-seq to detect genetic variations in the human GM12878 cell line. To increase coverage, filtered reads from the two replicates were combined for variant calling and subjected to the reference-dependent Read deconvolution step described above. Genetic variants were identified using gatk pipeline (McKenna et al. 2010) following the recommended best practice workflow. The resulting variants were benchmarked against the variants obtained using the NA12878 whole genome sequencing dataset (WGS, performed by JIMB NIST project). The number of true positive, false positive and false negative variants found using Methyl-SNP-seq were derived from the comparison between the two datasets.

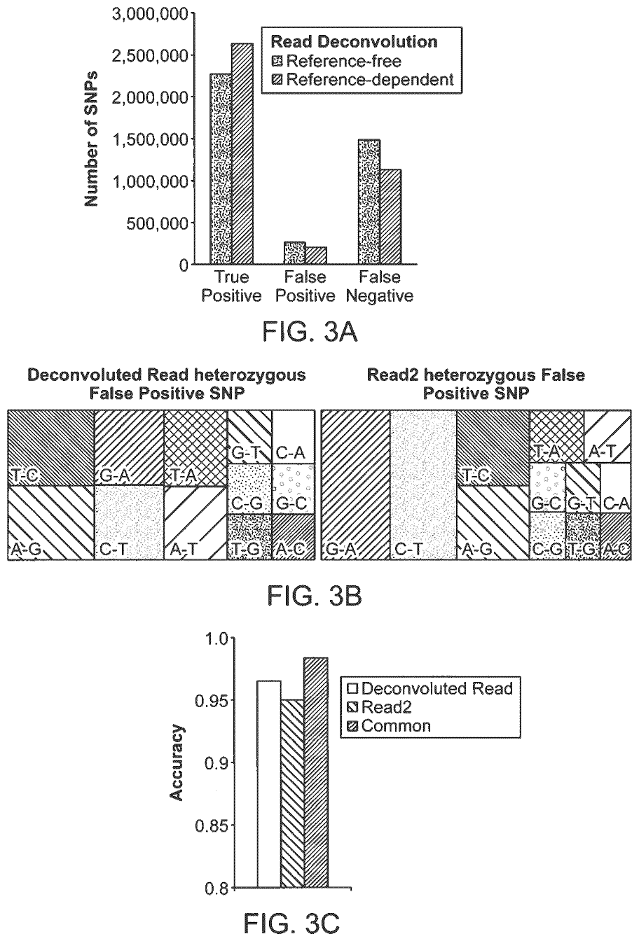

We first confirmed that the reference-dependent base calibration increases the number of true positive SNPs and reduces the number of false positive SNPs compared to reference-free base calibration (FIG. 3A)

Since both Deconvoluted Read and Read2 represent the original genome sequence, we can call genetic variants using either one. Overall, variants found using either the deconvoluted Read or Read2 shows a high level of agreement: 94% of SNPs found with the deconvoluted Read being identical to those found with Read 2 (FIG. 3A). According to the experimental design, as expected the Deconvoluted Read and Read2 had different types of false positive errors (FIG. 3B). Consequently, by using the common set of variants defined by both deconvoluted read and Read2, we could correct the variant calling error and improve the accuracy (FIG. 3C). Therefore, we chose the common variants between Deconvoluted read and Read2 as the Methyl-SNP-seq defined genetic variants.

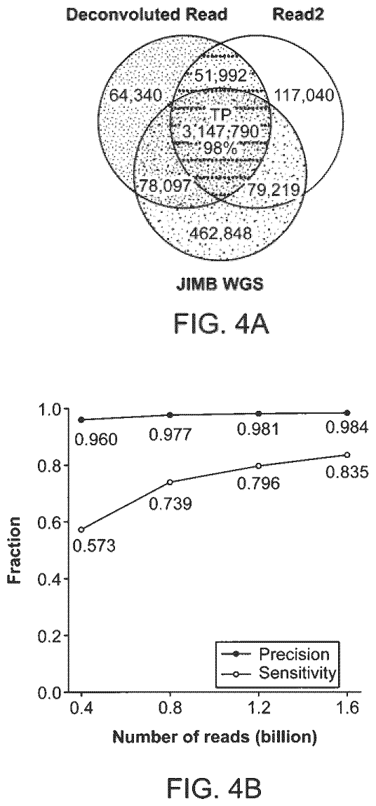

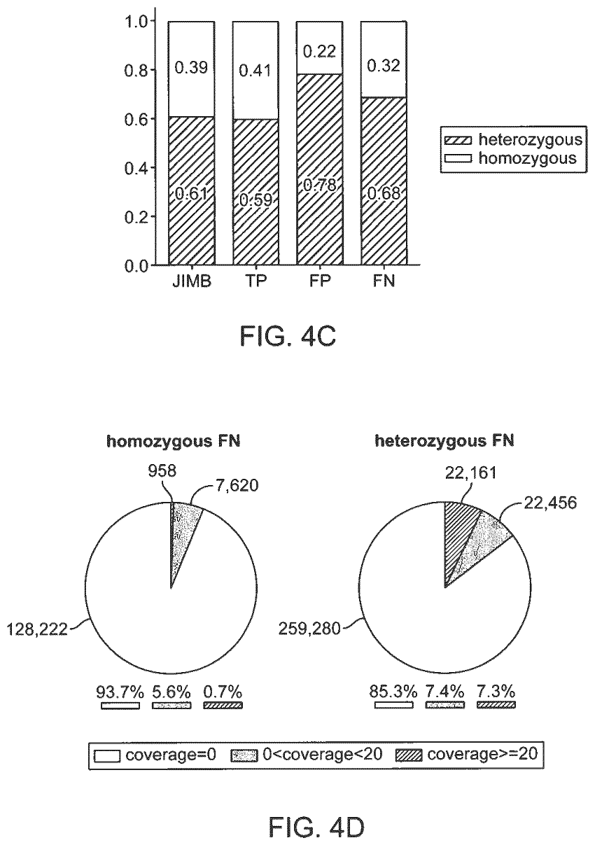

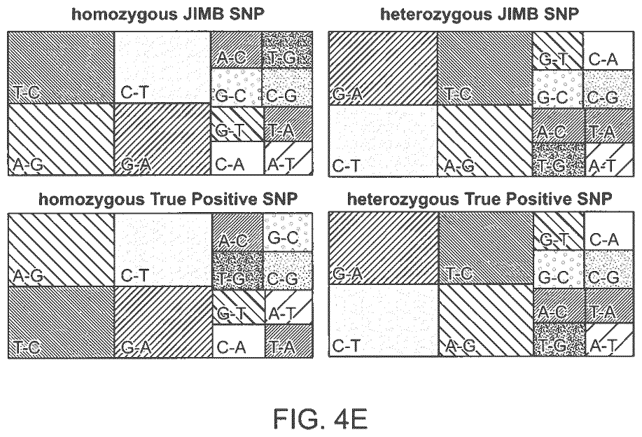

Using this set of common variants, we found 1,297,519 and 1,901,039 homozygous and heterozygous SNPs respectively. 98% of the common SNPs were also confirmed by WGS with a better agreement for homozygous SNPs (accounting for a fifth of the total false positive SNPs) compared to heterozygous SNPs (accounting for four fifth of the total false positive SNPs) (FIGS. 4A and 4C). As for the indel, our method also has a high accuracy with 94% agreement with WGS. These levels of agreement are comparable to the level of agreements typically observed between standard WGS (Zook et al. 2014)

We also performed the standard quality controls for variant calling and found that both Methyl-SNP-seq and WGS dataset displayed comparable metrics for both SNPs and indels (not shown). More specifically, the ratio of transition (Ti) to transversion (Tv) mutations is around 2.06 for both datasets demonstrating that both sets are unlikely to have a bias affecting the transition transversion ratio. As expected, we have less accuracy in detecting the C-T (REF-ALT in vcf), T-C, G-A and A-G type SNPs, which, combined, account for most of the errors (FIG. 3B).

To assess the sensitivity of Methyl-SNP-seq in identifying variants, we randomly sampled different numbers of reads. At equivalent coverage, we detected more than 80% of the WGS SNPs. This number drops to 60% when using only 25% of reads (FIG. 4B). We noted that the lack of read coverage was the major cause of these false negative SNPs that were not detected by Methyl-SNP-seq (FIG. 4D). Although having the same number of reads, the number of read bases was fewer in deconvoluted reads (109 billion) compared to WGS (160 billion), which was due to the shorter read length after trimming. Notably, reducing the number of reads used did not affect the accuracy for variant detection.

### Example 6: Methyl-SNP-seq Accurately Detects and Quantifies Cytosine Methylation at Base Resolution

We next evaluated the performance of Methyl-SNP-seq in identifying and quantifying cytosine methylation. The methylation status of individual cytosine was determined in the Read Deconvolution step and was added in the mapped bam file so that the base-resolution methylation information can be calculated using the conventional methylation calling tools such as bismark (Krueger and Andrews 2011).

Using the unmethylated Lambda spiked-in control we estimated the bisulfite conversion rate of Methyl-SNP-seq to be 97.5%. Overall CpG methylation is at 45% whereas CHG and CHH contexts methylation is on average 2.3% and 2.4% respectively. The GC bias of Methyl-SNP-seq follows closely the known GC bias observed for bisulfite sequencing with a preferential sequencing of AT rich genomic regions. Both replicates show comparable results (not shown).

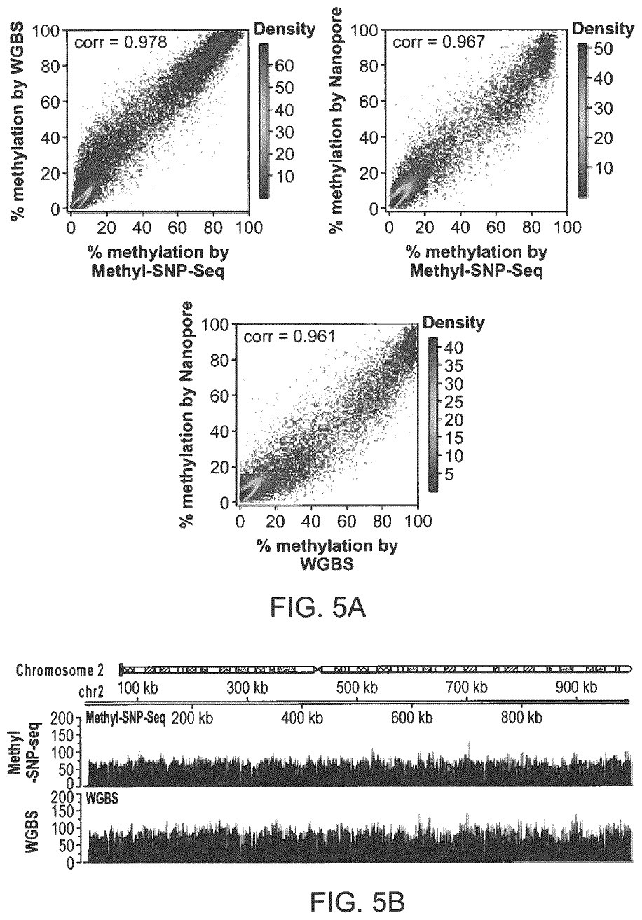

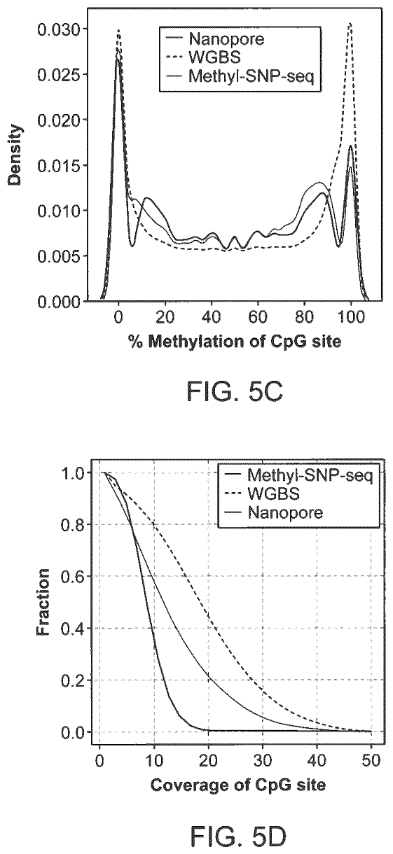

We subsequently measured the methylation level of CpG sites. We benchmarked our method's performance for modification profiling against two reference datasets generated by standard whole genome bisulfite sequencing method (ENCODE WGBS) and Nanopore sequencing (Jain et al. 2018). With 1.6 billion reads from the two replicates combined, we acquired 54 million CpG sites, for which 45 million had at least 5× coverage. These numbers are comparable with that of the WGBS method down-sampled to the similar number of reads (53 millions sites with >=5× coverage) (FIG. 5D). The genome-wide methylation level of CpG sites identified by Methyl-SNP-seq displays a bimodal distribution similar to those of the WGBS and Nanopore datasets (FIG. 5C) with a distribution that better resembles the Nanopore dataset. This result agrees with the observation that bisulfite sequencing overestimates the global methylation (Jain et al. 2018; Olova et al. 2018) (Ji et al. 2014).

Methylation patterns of CpG islands have been shown to affect gene expression and are linked to disease phenotypes (Robertson 2005). Therefore, we calculated the methylation level of the known CpG islands across the human genome and compared them between the three methods. We restricted our comparison to CpG islands with at least 50× coverage. The correlation results, as shown in FIG. 5A, demonstrated that Methyl-SNP-seq is highly correlated with both the ENCODE WGBS (Pearson correlation=0.98) and Nanopore (Pearson correlation=0.97) datasets (FIG. 5A and 5B). These results indicate that Methyl-SNP-seq is a highly accurate method for cytosine methylation quantification.

### Example 7: Allele-Specific Methylation Using Methyl-SNP-seq

Attempts to infer SNPs from WGBS have been previously published (Liu et al. 2012a) but require prohibitive genome wide coverage levels (e.g. >30× coverage required by Bis-SNP) to assess independently paired-end reads. This is because the identification of transition SNP such as C/T, G/A, A/G and T/C are confounded by the deamination step (Liu et al. 2012b). In contrast, our method can confidently distinguish cytosine methylation from an original transition SNPs along with other SNP types. Indeed, by using the redundancy of the double stranded DNA to read methylation and sequence from the same original DNA molecule, our method identifies both the methylation state and variants at single molecule level. This allows phasing of the methylation state with heterogeneous SNP directly on the read, enabling the identification of differentially methylated genomic regions (DMR) that are allele specific (ASDMR).

Using the whole genome Methyl-SNP-seq experiment done on human GM12878 described above, we identified a total of 34,909 ASDMR genomewide. An example of a known ASDMR (Suzuki et al. 2018; Kaplow et al. 2015) containing the heterozygous SNP rs11686156 on chromosome 2 was analyzed. Among all the identified ASDMRs, 47% have SNP directly affecting CpG sites. This result is consistent with a previous study (Shoemaker et al. 2010), which reported that 38% to 88% of ASM regions are solely due to the presence of SNPs at CpG dinucleotides and indicates variation at CpG sites is a dominating factor for ASDMR. In this case, SNP not only disrupts the methylation pattern of the affected CpG site but also affects the methylation pattern of the neighboring regions. Therefore, CpG-SNPs are very important for DMR studies because they may play a role in the establishment of certain types of DMRs such as ASDMRs.

Allele specific methylation is also often associated with gene imprinting. Using a set of ASDMRs that are reported to be associated with known imprinted gene clusters in the human genome as reference (Fang et al. 2012), we were able to identify 24 ASDMRs at or near the reported imprinting control DMRs for 15 out of the 30 imprinted gene clusters (not shown). For example, we detected 2 ASDMRs overlapping with the known imprinted cluster of the GNAS gene (not shown). These two ASDMRs span a 17.8 kb region and include 670 CpG pairs. When examining the methylation level of all the CpGs within this region, we saw that most of the CpG sites have an average methylation level close to 50% whereas CpG sites in the flanking regions have elevated methylation levels, suggesting this entire 17.8 kb region is likely an imprinted DMR (not shown).

Allele specific methylation (ASM) is also known to be associated with X chromosome inactivation in female cells via regulating the X-inactive specific transcript (XIST) gene (Wutz 2011; Fang et al. 2012). Accordingly, our method detected several ASM near the XIST gene in the human lymphocyte cell GM12878 (female) (not shown). In addition, we also detected ASMs in the promoter regions of genes which are known to be subject to X-chromosome inactivation (XCI) (Cotton et al. 2015)(Sharp et al. 2011) such as PDK3 and MBTPS2 (not shown).

Previous study (Kaplow et al. 2015) found that genomic regions with chromatin states consistent with active transcription and active enhancers were enriched for CpGs with mQTLs (ASM), suggesting that some of these ASM may affect transcription or enhancer activity. In our study, we found that CpGs that are associated with ASDMRs are significantly enriched, compared to random CpG regions, in enhancers which includes both active and primed enhancers and are marked by histone H3K4me1 modification in the absence of histone H3K4me3 modification (χ2=98.3, df=1, P-value<1e-9, fold change=1.5)

However, ASDMR CpGs are not enriched in active enhancers identified by H3K27Ac modification (fold change=0.9). In addition, ASDMR CpGs are significantly depleted in the promoter regions marked with histone H3K4me3 modification (χ2=120.1, df=1, P-value<1e-9, fold change=0.7). Interestingly, ASDMR CpGs are also enriched in the genomic regions with repressive histone mark H3K9me3 (χ2=29.1, df=1, P-value=6.8E-8, fold change=1.4). This histone mark is associated with heterochromatin and frequently coexists with DNA methylation. H3K9me3 is also reported to play a role in establishing imprinted X-chromosome inactivation in mice (Fukuda et al. 2014).

### Example 8: Methyl-SNP-seq can be Performed in Conjunction with the Conventional Probe-Based Target Enrichment

While providing a comprehensive view of the human genome, whole genome sequencing remains cost-prohibitive for analyzing a large number of clinical samples. In contrast, targeted sequencing with a focus on specific regions of interest is more widely and commonly used. In particular, targeted Bisulfite Sequencing is designed to measure site-specific DNA methylation changes. Accordingly, it normally requires design of specific bait probes capturing the bisulfite converted regions. Unlike the conventional targeted bisulfite sequencing, Methyl-SNP-seq method contains the original genome sequence (FIG. 1A) that can hybridize to the standard bait probes. Thus, in theory Methyl-SNP-seq can be easily adapted to the conventional targeted enrichment method with any standard probe sets.

To demonstrate the applicability of Methyl-SNP-seq for target enrichment, we tested Methyl-SNP-seq combined with the Twist human comprehensive exome panel. The targeted Methyl-SNP-seq had a high mapping efficiency with 96% of read mapping to the human genome. Bisulfite conversion rate is also very high, with 97% conversion at CHG and CHH contexts (not shown). Targeted-Methyl-SNP-seq also showed comparable target enrichment capability (not shown) compared to the standard exome targeted capture with the exception of that it had lower coverage of AT rich regions (AT_DROPOUT=9). We also applied stringent filters to remove PCR duplicates etc., consequently having about 11 million deconvoluted reads from two replicates. Although the Twist exome panel is designed to capture the gene body rather than the promoter regions, we still found 11783 CpG islands captured with coverage above 50. Like the whole genome sequencing, the methylation quantification of these CpG islands was reliable, consistent with the WGBS and Nanopore analysis (not shown). As for the variant detection, using the same probe panel at equivalent read depth (about 10 million reads used for variant calling), the precision of targeted Methyl-SNP-seq sequencing (precision=0.8) was lower to the standard targeted sequencing (precision=0.9) (not shown) (Zhou et al. 2021).

### Example 9: Reference-Free identification of m5C in Bacteria Using Methyl-SNP-seq

Another application of Methyl-SNP-seq is on the identification of methylation in organisms for which a reference genome or assembly is missing. This is often the case for environmental samples and microbiomes. In these cases, conversion-based methods to call methylation (e.g. bisulfite sequencing) cannot be used because these methods rely on differentiating between a genuine T and a C to T conversions using a reference genome. The Methyl-SNP-seq method, on the other hand, identifies cytosine methylation directly on the paired-end reads in a reference independent manner. Additionally, it reports methylation status of individual cytosine sites with sequence context information at single base resolution and at single molecule level, which is most suitable for methylation motif studies. Furthermore, our Methyl-SNP-seq method also reports the original genomic sequences that can be used for genome assemblies of a single organism or a mixed population.

To demonstrate the effectiveness of Methyl-SNP-seq for these applications, we performed Methyl-SNP-seq using genomic DNA of an isolated strain of E. coli K12). We first investigated whether we can assemble the deconvoluted reads into a reliable reference genome. Using the Velvet assembler (Zerbino 2010), we obtained a good assembly from the E. coli data (16 million deconvoluted reads) with high genome coverage (94% of the genome covered) and high sequence identity (2.21 mismatches per 100 kbp) (not shown), which was comparable to the performance of the assembler for single end short read assembly using standard DNA-seq.

We determined the methylase specificity of the bacteria directly on the deconvoluted reads without mapping. To achieve this, we randomly selected 0.3 deconvoluted reads and counted the number of occurrences of all the 8bp kmers (8mers) having methylated or unmethylated cytosine from these reads. We applied a Binomial model with Bonferroni adjustments to identify the 8bp sequence having significantly higher methylation level. These sequences were further grouped using Hierarchical clustering to uncover the consensus methylated motif indicative of methylase specificity(ies).

Two context clusters were found from 128 significantly enriched 8mers, CCAGG and CCTGG which can be further combined into CCWGG (with W=A or T) to reveal the correct specificity for the E. coli dcm methylase (Marinus and Morris 1973; May and Hattman 1975) (not shown).

We also perform Methyl-SNP-seq on a mixed sample consisting of genomic DNA of two bacterial strains (E. coli K12 and Clostridium ABKn8 strains) to mimic a simple mixed bacteria population. Using 0.6 million deconvoluted reads we found 3 motifs instead of the 2 motifs expected for these strains (not shown). These motifs correspond to CCWGG, GCNNGC and CGWCG. The first two motifs match the expected methylase specificities of E. coli dem and C. acetobutylicum (Baum et al. 2021), respectively. The third motif, CGWCG, however, was unexpected. To investigate the origin of this methylated motif, we assembled the deconvoluted reads of the mixed sample library and used the 100 longest assembled contigs as reference to determine methylation motifs. All of the 100 contigs contain a single methylated motif, among which 36 and 62 have the CCWGG motif and the GCNNGC motif, consistent with their E. coli and C. acetobutylicum origins (not shown). Two out of the 100 contigs have the unexpected CGWCG motif and both contigs have high sequence homology to the genome sequence of a Bacillus strain by BLAST search, implying that there was a contamination and most likely a Bacillus strain in the mixed sample. We further annotated the assembled contigs which contain the CGWCG motif using Prokka and identified a single gene with cytosine-specific DNA methylase domain. This suggests the contamination strain is methylated. Additionally, the methylation analysis using EM-seq confirmed the presence of a CGWCG methylation motif in the used Clostridium sample.

In this example, we demonstrated that Methyl-SNP-seq method can not only identify all the methylation motifs from a mixed sample in a reference independent manner, but can also resolve the composition of a mixed population by assembling the deconvoluted sequences and using methylation motif as a species/strain signature and genome binning criteria.

### Example 10: Methods Employing use of a Single Hairpin

This example describes a method for producing a deamination-resistant strand of DNA using one hairpin adaptor. An exemplary overview is shown in FIG. 6.

The double stranded DNA substrate is fragmented to lengths suitable for sequencing. A variety of fragmentation methods may be used (e.g., mechanical shearing, NEBNext UltraShear enzymatic fragmentation). The selected fragmentation method should not remove methylation marks. The implementation of the methods describe below may be adjusted to meet the needs of the selected sequencing system (e.g., sequencing systems from companies such as Illumina, Element, MGI, Nanopore, PacBio, Singular Genomics, etc.).

The strands of the fragmented double-stranded DNA are separated to create single stranded DNA. A variety of methods may be used for strand separation. Typical methods include treatment with heat, salt, and/or chemical conditions. Examples include adding formamide or sodium hydroxide to a final concentration of about 20%, mixing, and incubating at 85 degrees C. for about 10 minutes for formamide or fifty degrees C. for about 10 minutes for sodium hydroxide, then placing the sample on ice.

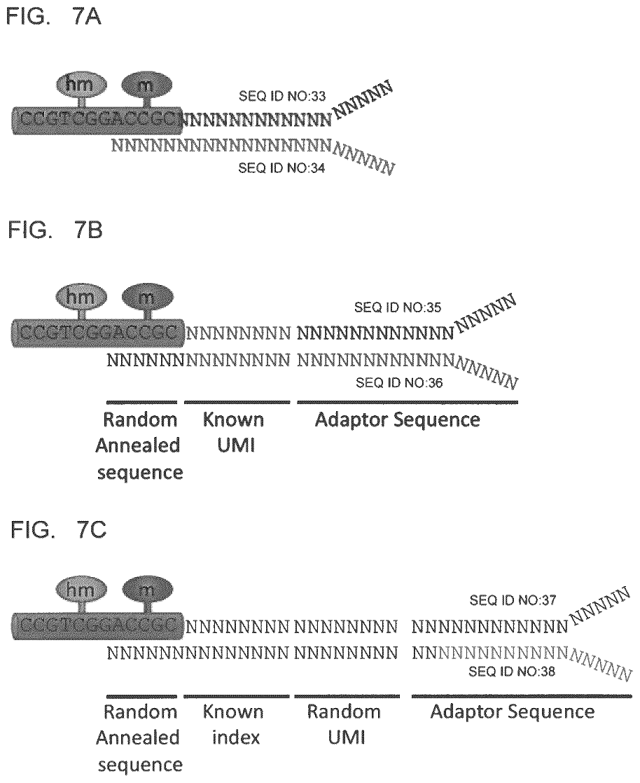

Sequencing adaptors are 3′ ligated to the resulting single stranded DNA. Adaptors can be ligated as double stranded or single stranded. For double-stranded ligation, the sequencing adaptors are annealed prior to ligation and have random nucleotides on the strand that does not ligate to the single stranded DNA. This random stretch of nucleotides may stabilize the ligation of the adaptor to the 3′ end of the single stranded DNA and is used as a primer to make a copy to produce a neosynthesized strand. See, for example, FIG. 7A.

The adaptor could also have an inline unique molecular identifier (UMI). The structure of the adaptor could include a mixture of known sequences for UMIs, that would be ligated to the single stranded DNA, or could be a random UMI flanked by known adaptor sequence and a known index sequence. See, for example, FIG. 7B and 7C.

As an example, the strand to be ligated could be treated as follows: 5′ end phosphorylation and 3′ end ddNTP. The non-ligated strand would be treated as follows: 5′ end phosphorothioate, ddNTP and 3′ end phosphorothioate.

The ligation method could be as follows, among any of a variety of other conditions: add fragmented DNA (e.g., 55 μl); 5 μM Annealed adaptor (e.g., 5 μl); ET SSB (optional) (e.g., 0.5 μl); Ligase Buffer (e.g., 6.5 μl); Ligase (e.g., 3 μl), ligase; incubate at 20° C. for 15 minutes.

For single stranded ligation of adaptor, the strand to be ligated could be treated as follows: 5′ end phosphorylation and 3′ end ddNTP.

Primer extension may then be performed. The non-annealed strand of the sequencing adaptor can be used for primer extension. This copies the original strand. Modified dCTP (e.g., 5mdCTP) are introduced where cytosines would have been located on the copied strand. This permits identification of the genetic sequence. An exemplary reaction mixture is Adaptor Annealed DNA (e.g., 65 μl); 10× Polymerase Buffer (e.g., 9 μl); 10 mM dTTP, dGTP, dATP, modified dCTP, e.g., 5mdCTP (e.g., 8 μl); water (e.g., 6 μl), Polymerase such as klenow or klenow exo minus (e.g., 2 μl); incubate at 37° C. for 15-30 min.

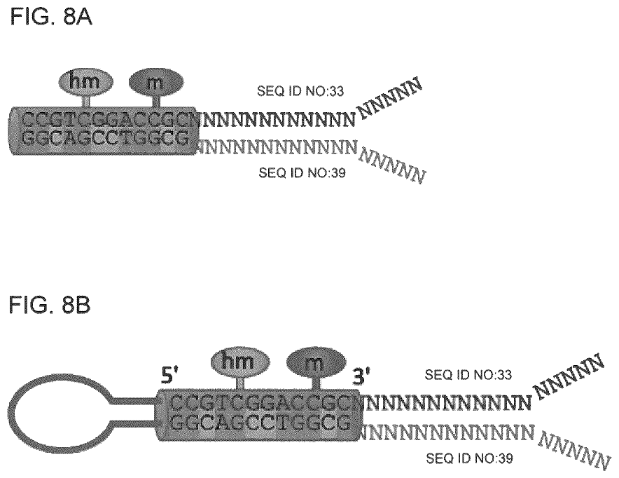

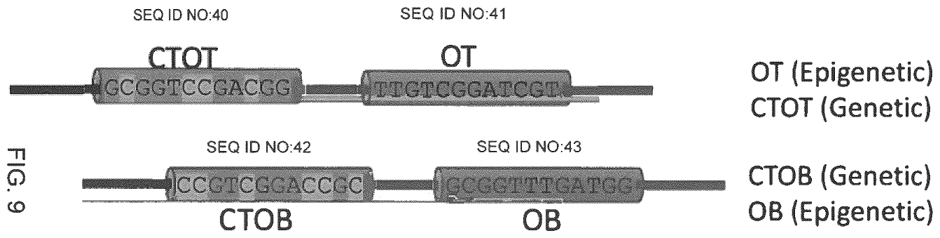

After primer extension the DNA is double stranded (containing the original sequence in a duplex with the neosynthesized sequence; see FIG. 8A) and may be cleaned-up (e.g., using column-based, bead-based purification method, or another method).

Hairpin adaptor may be prepped by annealing before use. This is a single stranded oligo with two complementary regions located at the 5′ end and at the 3′ end of the oligo. The oligo will form a hairpin structure and can be annealed to the primer extended DNA. Note, if klenow exo minus is used as the polymerase for primer extension, the extended strand will have an A overhang. The hairpin adaptor, could have an T overhang to reduce adaptor dimer formation. An exemplary reaction mixture is: Adaptor Annealed DNA (e.g., 30 μl); 10× Ligase buffer (e.g., 4 μl); 10 μM Annealed adaptor (e.g., 4 μl); and ligase (e.g., 2 μl). An alternative is ligation of linear double stranded DNA, instead of a hairpin adaptor, then use of TeIN (or another strategy) to circularize the end. After hairpin ligation (see FIG. 8B) the DNA may be cleaned up using column-based, bead-based purification, or any other method. As an example, the material may be eluted in 28 μl of water or buffer (e.g., 10 mM Tris pH 8.0).

Enzymatic conversion of cytosines (e.g., to uracils) is then performed. This can be done by enzymatic conversion or bisulfite conversion. The original single stranded DNA molecule contains both unmethylated and methylated cytosines. Conversion results in differentiation of the methylated and non-methylated cytosines. The copied strand contains only methylated cytosines (from use of modified dCTP). This represents the genetic information as the methylated cytosines will not be converted.

An exemplary approach is NEBNext E7120 Oxidation/Glucosylation, using a reaction mixture such as: Hairpin adaptor ligated DNA (e.g., 28 μl); TET2 Reaction Buffer (e.g., 10 μl); Oxidation Supplement (e.g., 1 μl); DTT (e.g., 1 μl); Oxidation Enhancer (e.g., 1 μl); TET2 (e.g., 4 μl). To this mixture, add 5 μl of 1:1250 dilution of 500 mM Fe (II); incubate at 37° C. for 1 hour; add 1 μl of Stop Solution; incubate at 37° C. for 1 hour. After the oxidation/glucosylation reaction the DNA may be cleaned up using column-based, bead-based or another purification method. Elute, for example, in 16 μl of elution buffer.

If required, the DNA can be denatured using any method (denaturation may not be required when using double stranded deaminase). For example, add to the Oxidized DNA (e.g., 16 μl) either formamide or 0.1 N sodium hydroxide (e.g., 4 μl) and incubate at 85° C. for 10 minutes, and then place on ice to cool. Cytosine deamination is then performed. For example, mix together and incubate the following mixture for 3 hours at 37° C.: Denatured DNA (e.g., 20 μl); water (e.g., 14 μl); APOBEC Reaction Buffer (e.g., 4 μl); BSA (e.g., 1 μl); APOBEC (e.g., 1 μl).

Library Amplification is then performed. A variety of methods may be used. For example, the deaminated DNA (e.g., 40 μl) is combined with EM-seq primers (e.g., 5 μl) and 2× Q5U polymerase (45 μl), and amplified under conditions such as: Initial Denaturation at 98 degrees C. for 30 seconds, 1 cycle; Denaturation at 98 degrees C. for 10 seconds, cycles depending on input; Annealing at 62 degrees C. for 30 seconds, cycles depending on input; Extension at 65 degrees C. for 60 seconds, cycles depending on input; and Final Extension at 65 degrees C. for 5 minutes, 1 cycle. Sequencing of the amplified DNA is then performed, and will give both epigenetic and genetic information. See FIG. 9.

### REFERENCES

Baum, Chloé, Yu-Cheng Lin, Alexey Fomenkov, Brian P. Anton, Lixin Chen, Bo Yan, Thomas C. Evans, Richard J. Roberts, Andrew C. Tolonen, and Laurence Ettwiller. 2021. “Rapid Identification of Methylase Specificity (RIMS-Seq) Jointly Identifies Methylated Motifs and Generates Shotgun Sequencing of Bacterial Genomes.” Nucleic Acids Research49 (19): e113.

Baylin, Stephen B., and Peter A. Jones. 2016. “Epigenetic Determinants of Cancer.” Cold Spring Harbor Perspectives in Biology8 (9). https://doi.org/10.1101/cshperspect.a019505.

Blow, Matthew J., Tyson A. Clark, Chris G. Daum, Adam M. Deutschbauer, Alexey Fomenkov, Roxanne Fries, Jeff Froula, et al. 2016. “The Epigenomic Landscape of Prokaryotes.” PLOS Genetics12 (2): e1005854.

Clark, Tyson A., Xingyu Lu, Khai Luong, Qing Dai, Matthew Boitano, Stephen W. Turner, Chuan He, and Jonas Korlach. 2013. “Enhanced 5-Methylcytosine Detection in Single-Molecule, Real-Time Sequencing via Tet1 Oxidation.” BMC Biology11 (January): 4.

Cleary, John G., Ross Braithwaite, Kurt Gaastra, Brian S. Hilbush, Stuart Inglis, Sean A. Irvine, Alan Jackson, et al. 2014. “Joint Variant and de Novo Mutation Identification on Pedigrees from High-Throughput Sequencing Data.” Journal of Computational Biology: A Journal of Computational Molecular Cell Biology21 (6): 405-19.

Cornish, Adam, and Chittibabu Guda. 2015. “A Comparison of Variant Calling Pipelines Using Genome in a Bottle as a Reference.” BioMed Research International2015 (October): 456479.

Cotton, Allison M., E. Magda Price, Meaghan J. Jones, Bradley P. Balaton, Michael S. Kobor, and Carolyn J. Brown. 2015. “Landscape of DNA Methylation on the X Chromosome Reflects CpG Density, Functional Chromatin State and X-Chromosome Inactivation.” Human Molecular Genetics24 (6): 1528-39.

Fang, Fang, Emily Hodges, Antoine Molaro, Matthew Dean, Gregory J. Hannon, and Andrew D. Smith. 2012. “Genomic Landscape of Human Allele-Specific DNA Methylation.” Proceedings of the National Academy of Sciences of the United States of America109 (19): 7332-37.

Feng, Hao, Karen N. Conneely, and Hao Wu. 2014. “A Bayesian Hierarchical Model to Detect Differentially Methylated Loci from Single Nucleotide Resolution Sequencing Data.” Nucleic Acids Research42 (8): e69.

Fukuda, Atsushi, Junko Tomikawa, Takumi Miura, Kenichiro Hata, Kazuhiko Nakabayashi, Kevin Eggan, Hidenori Akutsu, and Akihiro Umezawa. 2014. “The Role of Maternal-Specific H3K9me3 Modification in Establishing Imprinted X-Chromosome Inactivation and Embryogenesis in Mice.” Nature Communications5 (November): 5464.

Greenberg, Maxim V. C., and Deborah Bourc'his. 2019. “The Diverse Roles of DNA Methylation in Mammalian Development and Disease.” Nature Reviews. Molecular Cell Biology20 (10): 590-607.

Jain, Miten, Sergey Koren, Karen H. Miga, Josh Quick, Arthur C. Rand, Thomas A. Sasani, John R. Tyson, et al. 2018. “Nanopore Sequencing and Assembly of a Human Genome with Ultra-Long Reads.” Nature Biotechnology36 (4): 338-45.

Ji, Lexiang, Takahiko Sasaki, Xiaoxiao Sun, Ping Ma, Zachary A. Lewis, and Robert J. Schmitz. 2014. “Methylated DNA Is over-Represented in Whole-Genome Bisulfite Sequencing Data.” Frontiers in Genetics5 (October): 341.

Kaplow, Irene M., Julia L. Maclsaac, Sarah M. Mah, Lisa M. McEwen, Michael S. Kobor, and Hunter B. Fraser. 2015. “A Pooling-Based Approach to Mapping Genetic Variants Associated with DNA Methylation.” Genome Research25 (6): 907-17.

Krueger, Felix, and Simon R. Andrews. 2011. “Bismark: A Flexible Aligner and Methylation Caller for Bisulfite-Seq Applications.” Bioinformatics27 (11): 1571-72.

Langmead, Ben, and Steven L. Salzberg. 2012. “Fast Gapped-Read Alignment with Bowtie 2.” Nature Methods9 (4): 357-59.

Liang, Jialong, Kun Zhang, Jie Yang, Xianfeng Li, Qinglan Li, Yan Wang, Wanshi Cai, Huajing Teng, and Zhongsheng Sun. 2021. “A New Approach to Decode DNA Methylome and Genomic Variants Simultaneously from Double Strand Bisulfite Sequencing.” Briefings in Bioinformatics22 (6). https://doi.org/10.1093/bib/bbab201.

Liu, Yaping, Kimberly D. Siegmund, Peter W. Laird, and Benjamin P. Berman. 2012a. “Bis-SNP: Combined DNA Methylation and SNP Calling for Bisulfite-Seq Data.” Genome Biology13 (7): R61.

. 2012b. “Bis-SNP: Combined DNA Methylation and SNP Calling for Bisulfite-Seq Data.” Genome Biology13 (7): R61.

Marinus, M. G., and N. R. Morris. 1973. “Isolation of Deoxyribonucleic Acid Methylase Mutants of Escherichia Coli K-12.” Journal of Bacteriology114 (3): 1143-50.

May, M. S., and S. Hattman. 1975. “Analysis of Bacteriophage Deoxyribonucleic Acid Sequences Methylated by Host-and R-Factor-Controlled Enzymes.” Journal of Bacteriology123 (2): 768-70.

Mckenna, Aaron, Matthew Hanna, Eric Banks, Andrey Sivachenko, Kristian Cibulskis, Andrew Kernytsky, Kiran Garimella, et al. 2010. “The Genome Analysis Toolkit: A MapReduce Framework for Analyzing next-Generation DNA Sequencing Data.” Genome Research20 (9): 1297-1303.

Olova, Nelly, Felix Krueger, Simon Andrews, David Oxley, Rebecca V. Berrens, Miguel R. Branco, and Wolf Reik. 2018. “Comparison of whole-genome bisulfite sequencing library preparation strategies identifies sources of biases affecting DNA methylation data.” Genome Biology19 (1): 33.

Rand, Arthur C., Miten Jain, Jordan M. Eizenga, Audrey Musselman-Brown, Hugh E. Olsen, Mark Akeson, and Benedict Paten. 2017. “Mapping DNA Methylation with High-Throughput Nanopore Sequencing.” Nature Methods14 (4): 411-13.

Robertson, Keith D. 2005. “DNA Methylation and Human Disease.” Nature Reviews. Genetics6 (8): 597-610.

Sharp, Andrew J., Elisavet Stathaki, Eugenia Migliavacca, Manisha Brahmachary, Stephen B. Montgomery, Yann Dupre, and Stylianos E. Antonarakis. 2011. “DNA Methylation Profiles of Human Active and Inactive X Chromosomes.” Genome Research21 (10): 1592-1600.

Shoemaker, Robert, Jie Deng, Wei Wang, and Kun Zhang. 2010. “Allele-Specific Methylation Is Prevalent and Is Contributed by CpG-SNPs in the Human Genome.” Genome Research20 (7): 883-89.

Simpson, Jared T., Rachael E. Workman, P. C. Zuzarte, Matei David, L. J. Dursi, and Winston Timp. 2017. “Detecting DNA cytosine methylation using nanopore sequencing.” Nature Methods14 (4): 407-10.

Suzuki, Masako, Will Liao, Frank Wos, Andrew D. Johnston, Justin DeGrazia, Jennifer Ishii, Toby Bloom, Michael C. Zody, Soren Germer, and John M. Greally. 2018. “Whole-Genome Bisulfite Sequencing with Improved Accuracy and Cost.” Genome Research28 (9): 1364-71.

Tourancheau, Alan, Edward A. Mead, Xue-Song Zhang, and Gang Fang. 2021. “Discovering Multiple Types of DNA Methylation from Bacteria and Microbiome Using Nanopore Sequencing.” Nature Methods18 (5): 491-98.

Wilbanks, Elizabeth G., Hugo Dore, Meredith H. Ashby, Cheryl Heiner, Richard J. Roberts, and Jonathan A. Eisen. 2022. “Metagenomic Methylation Patterns Resolve Bacterial Genomes of Unusual Size and Structural Complexity.” The ISME Journal, April. https://doi.org/10.1038/s41396-022-01242-7.

Wutz, Anton. 2011. “Gene Silencing in X-Chromosome Inactivation: Advances in Understanding Facultative Heterochromatin Formation.” Nature Reviews. Genetics12 (8): 542-53.

Zerbino, Daniel R. 2010. “Using the Velvet de Novo Assembler for Short-Read Sequencing Technologies.” Current Protocols in Bioinformatics/Editoral Board, Andreas D. Baxevanis . . . [et Al.]Chapter 11 (September): Unit 11.5.

Zhou, Juan, Mancang Zhang, Xiaoqi Li, Zhuo Wang, Dun Pan, and Yongyong Shi. 2021. “Performance Comparison of Four Types of Target Enrichment Baits for Exome DNA Sequencing.” Hereditas158 (1): 10.

Zook, Justin M., Brad Chapman, Jason Wang, David Mittelman, Oliver Hofmann, Winston Hide, and Marc Salit. 2014. “Integrating Human Sequence Data Sets Provides a Resource of Benchmark SNP and Indel Genotype Calls.” Nature Biotechnology32 (3): 246-51.

1. A method for generating a deamination-resistant strand of DNA, comprising
(a) ligating a hairpin adaptor to a double-stranded fragment of DNA to produce a ligation product;
(b) enzymatically generating a free 3′ end in a double-stranded region of the hairpin adaptor in the ligation products; and
(c) extending the free 3′ end in a dCTP-free reaction mix that comprises a strand-displacing or nick-translating polymerase, dGTP, dATP, dTTP and modified dCTP to generate a hairpin product that has an original strand and a neosynthesized strand that contains modified Cs.

2. The method of claim 1, further comprising
(d) deaminating the hairpin product or an adaptor-ligated product thereof, wherein the modified Cs protect the neosynthesized strand from deamination.

3. The method of claim 2, wherein the deaminating is done using bisulfite or using a cytosine deaminase, optionally after enzymatically protecting any modified Cs in the original strand from deamination.

4. (canceled)

5. The method of claim 3, wherein the cytosine deaminase modifies a double-stranded or single-stranded substrate.

6. The method of claim 2, further comprising amplifying the deaminated product of step (d) thereby converting any deaminated Cs to Ts in the amplification product.

7. The method of claim 6, further comprising enriching for target molecules using a probe that is complementary to a sequence in the double-stranded fragment of (a).

8. The method of claim 2, further comprising sequencing the deaminated product, or an amplification product thereof, to produce sequence.

9. The method of claim 8, further comprising identifying a C in the sequence corresponding to the original strand, wherein the C corresponds to a modified cytosine.

10. The method of claim 9, further comprising mapping the modified cytosine to a site in a reference genome and annotating the site as being modified.

11. The method of claim 1, wherein the modified dCTP is dmCTP, pyrrolo-dCTP or N⁴-dmCTP.

12. The method of claim 1, wherein the double-stranded fragment of DNA is selected from a fragment of mammalian DNA and a molecule of cfDNA.

13. (canceled)

14. The method of claim 1, further comprising enzymatically modifying the double-stranded fragment of DNA, the ligation product or hairpin product to protect any modified cytosines or hydroxymethylcytosines from deamination.

15. The method claim 1, wherein in step (a) both ends of the double-stranded fragment of DNA are ligated to the hairpin adaptor and in step (b) the top and bottom strands of the double-stranded fragment of DNA become separated.

16. The method of claim 1, wherein step (b) is done using USER, an endonuclease, a nicking endonuclease or an RNase.

17. The method of claim 1, wherein the hairpin adaptor has at least one modified C and no Cs.

18. The method of claim 1, wherein the modified C of the adaptor is mCTP, pyrrolo-CTP or N⁴-mCTP.

19. A reaction mix comprising:
(a) a hairpin DNA that has a free 3′ end in a double-stranded region;
(b) a strand-displacing or nick-translating polymerase, and
(c) dGTP, dATP, dTTP, modified dCTP and no dCTP.

20. The reaction mix of claim 19, wherein the hairpin DNA comprises a fragment of mammalian DNA ligated to a hairpin adaptor or comprises a molecule of cfDNA ligated to a hairpin adaptor.

21. (canceled)

22. The reaction mix claim 19, wherein the modified dCTP is dmCTP, pyrrolo-dCTP or N⁴-dmCTP.

23. A nucleic acid molecule comprising, in order from 5′ to 3′:
a first sequence, a linker, and a second sequence, wherein:
(a) the first sequence is composed of Gs, As, Ts, Cs and modified Cs;
the second sequence is composed of Gs, As, Ts, modified Cs and no Cs; and
the first and second sequences are complementary; or
(b) the first sequence is composed of Gs, As, Ts, Us and modified Cs and
the second sequence is composed of Gs, As, Ts, modified Cs and no Cs; and
the first and second sequences are complementary except for the Us in the first sequence.

24. (canceled)

25. A kit for generating a deamination-resistant strand of DNA, comprising:
(a) a hairpin adaptor containing a U in a double-stranded region of the adaptor;
(b) one or more enzymes that create a nick at the site of the U;
(c) a modified dCTP; and
(d) a nick-translating or strand-displacing polymerase, optionally wherein the modified dCTP is dmCTP, pyrrolo-dCTP or N⁴-dmCTP.

26. (canceled)

27. The kit of claim 25, wherein the adaptor contains modified Cs and no Cs, and optionally wherein the modified Cs of the adaptor are mCTP. pyrrolo-CTP or N⁴-mCTP.

28. (canceled)

29. The kit of claim 25, further comprising a deaminase, wherein the modified Cs are deamination resistant.

30. A method for generating a deamination-resistant strand of DNA, comprising:
(a) separating the strands of a double-stranded fragment of DNA to produce a single-stranded fragment;
(b) attaching a double-stranded adaptor to the 3′ end of the single-stranded fragment;
(c) extending the free 3′ end of an attached double-stranded adaptor in a dCTP-free reaction mix that comprises a strand-displacing or nick-translating polymerase; and dGTP, dATP, dTTP, and modified dCTP, to generate a double-stranded product, and
(d) attaching a hairpin adaptor to the 5′ end of the double-stranded product to generate a hairpin product that has an original strand and a neosynthesized strand that contains modified Cs.
